(19) United States
(12) Patent Application Publication
(10) Pub. No.: US 2024/0392298 A1
(43) Pub. Date: Nov. 28, 2024

(54) BIOLOGICALLY STABLE XNAZYME THAT EFFICIENTLY SILENCES GENE EXPRESSION IN CELLS

(71) Applicant: THE REGENTS OF THE UNIVERSITY OF CALIFORNIA, OAKLAND, CA (US)

(72) Inventors: John C. Chaput, IRVINE, CA (US); Yajun Wang, IRVINE, CA (US); Robert Spitale, IRVINE, CA (US); Kim T. Nguyen, IRVINE, CA (US)

(73) Assignee: THE REGENTS OF THE UNIVERSITY OF CALIFORNIA, OAKLAND, CA (US)

(21) Appl. No.: 18/260,166

(22) PCT Filed: Dec. 23, 2021
(86) PCT No.: PCT/US2021/065063

§ 371 Date: Jun. 30, 2023

Related U.S. Application Data
(60) Provisional application No. 63/132,351, filed on Dec. 30, 2020.

(51) Int. Cl. C12N 15/113

(52) U.S. Cl. CPC C12N 15/1137; C12Y 306/05002; C12N 2310/127; C12N 2310/322; C12N 2310/3231

(57) ABSTRACT

Efforts to use RNA-cleaving DIMA enzymes (DNAzymes) as gene silencing agents in therapeutic applications have stalled due to their low efficacy in clinical trials. Here the present invention reports a xeno-nucleic acid (XNA) modified version of the classic DNAzyme 10-23 that achieves multiple turnover activity under cellular conditions and resists nuclease digestion. The new reagent overcomes the problem of product inhibition limiting previous 10-23 designs using molecular chemotypes with DNA. FANA, and TNA backbone architectures that balance the effects of enhanced biological stability with RNA hybridization and divalent metal ion coordination. In cultured mammalian cells. X 10-23 facilitates persistent gene silencing by efficiently degrading exogenous and endogenous mRNA transcripts. Together, these results demonstrate that new molecular chemotypes can improve the activity and stability of DNAzymes, and may provide a new route for nucleic acid enzymes to reach the clinic.

### CROSS-REFERENCES TO RELATED APPLICATIONS

This application claims benefit of U.S. Provisional Application No. 63/132,351 filed Dec. 30, 2020, the specification(s) of which is/are incorporated herein in their entirety by reference.

### REFERENCE TO A SEQUENCE LISTING

The content of the ASCII text file of the electronically submitted sequence listing named “UCI013_Seq”, which is 25 kb in size and was created on Jan. 11, 2024, is incorporated herein by reference in its entirety.

### FIELD OF THE INVENTION

The present invention relates to 10-23 deoxyribonucleic acid enzyme (DNAzyme) analog compositions and methods of use such as, for example, to efficiently silence gene expression in cells.

### BACKGROUND OF THE INVENTION

The DNA enzyme (DNAzyme) 10-23 (FIG. 1A), also known as Dz10-23 or simply 10-23, is the best characterized example of a Mg²⁺-dependent RNA-cleaving DNA enzyme created by in vitro selection. The enzyme was identified from a population of 10¹⁴ unique DNA molecules using a stringent selection strategy that was designed to favor the enrichment of individual molecules that promote the site-specific cleavage of RNA transcripts. The enzyme comprises a 15-nucleotide (nt) catalytic domain that is flanked on both sides by substrate binding arms that can vary in length depending on the sequence of the RNA substrate. As with other oligonucleotide therapeutics, the RNA target is recognized by complementary Watson-Crick base pairing. Upon binding, RNA cleavage ensues at a predefined purine-pyrimidine (R-Y) junction with the highest activity levels observed for G-U dinucleotides. The cleavage mechanism involves metal-assisted deprotonation of a 2′-hydroxyl from the purine (R) nucleotide, followed by nucleophilic attack on the neighboring phosphodiester bond to yield an upstream cleavage product with a 2′,3′-cyclic phosphate and a downstream cleavage product with a 5′-hydroxyl group.

Over the years, 10-23 has been chemically modified in various ways to achieve improved efficacy in vivo and in cells. Chemical modifications used for this purpose include phosphorothioate linkages, 2′-O-methylribonucleotides, inverted 3′-3′ thymidine nucleotides, phosphoramidite linkages, and locked nucleic acids (LNA). The effect of these modifications on the catalytic activity of the enzyme ranges from deleterious to beneficial depending on the residue location and type of chemical modification. Most chemical modifications have been directed to the substrate binding arms with the goal of increasing the affinity of the reagent for the RNA target. However, this strategy poses a barrier to improving the catalytic activity of DNAzymes, as the modifications chosen for enhanced RNA binding often lead to product inhibition with the enzyme-product complex failing to dissociate from the post-catalytic state. Thus, new molecular designs are needed for DNAzymes to efficiently cleave mRNA transcripts in cellular systems.

### BRIEF SUMMARY OF THE INVENTION

It is an objective of the present invention to provide for compositions and methods that utilize xeno-nucleic acids (XNAs) that allow for the creation of 10-23 analog compositions with improved catalytic turnover and elevated biological stability, as specified in the independent claims. Embodiments of the invention are given in the dependent claims. Embodiments of the present invention can be freely combined with each other if they are not mutually exclusive.

In some embodiments, the present invention features a composition for gene silencing. In some embodiments, the composition comprises a 15-nucleotide catalytic domain according to SEQ ID NO: 1, a first substrate recognition domain 5′ to the catalytic domain, a second substrate recognition domain 3′ to the catalytic domain, a 5′ terminal threose nucleic acid (TNA) residue and a 3′ terminal TNA residue. In some embodiments, the composition comprises a catalytic domain. In some embodiments, one or more nucleic acids of the catalytic domain are replaced by xeno-nucleic acids (XNA). In other embodiments, one or more nucleic acids of the first substrate recognition domain or the second recognition domain is replaced by XNA. In other embodiments, the composition has enhanced stability and enhanced catalytic activity compared to a control molecule comprising wild type SEQ ID NO: 1 as its catalytic domain.

In other embodiments, the present invention may also feature a method of treating a disease or condition or a symptom thereof. In some embodiments, the method comprises administering an effective amount of a 10-23 analogue composition to a subject in need thereof. In some embodiments, the composition comprises a 15-nucleotide catalytic domain according to SEQ ID NO: 1, a first substrate recognition domain 5′ to the catalytic domain, a second substrate recognition domain 3′ to the catalytic domain, a 5′ terminal threose nucleic acid (TNA) residue and a 3′ terminal TNA residue. In some embodiments, the composition comprises a catalytic domain. One or more nucleic acids of the catalytic domain may be replaced by xeno-nucleic acids (XNA). In other embodiments, one or more nucleic acids of the first substrate recognition domain or the second recognition domain is replaced by XNA.

Without wishing to limit the present invention to any theory or mechanism, it is thought that xeno-nucleic acids (XNAs) offered a new molecular chemotype with physicochemical properties that could achieve enhanced biological stability without sacrificing catalytic activity under multiple turnover conditions that typify intracellular conditions. Guided by nucleic acid chemistry, the present invention searched for XNA residues that would provide a balanced solution to the problem of how to enhance the substrate binding kinetics while avoiding the harmful effects of product inhibition. Biological stability and catalytic turnover were the main obstacles separating DNAzymes from protein-catalyzed gene silencing reagents, such as antisense or siRNA reagents.

A typical DNAzyme has a catalytic core of 15 deoxyribonucleotides (SEQ ID NO: 1) flanked on both ends by substrate recognition domains. One of the first DNAzymes to be discovered was DNAzyme 10-23. Despite its enormous potential, DNAzymes have suffered from poor pharmacokinetics due to limited biological stability and poor catalytic activity under physiological concentrations of Mg⁺² ions.

The present invention features a reengineered version of the classic 10-23 DNAzyme that mediates persistent gene silencing activity in cultured mammalian cells, while simultaneously resisting nuclease digestion. The new reagent, termed X10-23 was discovered using a medicinal chemistry approach that probed each position in the DNA backbone for structural mutations that promote enhanced catalytic activity under simulated physiological conditions. The present results demonstrate that new molecular chemotypes can greatly improve the catalytic activity of a highly evolved DNAzyme, suggesting that molecular design is a powerful approach for optimizing nucleic acid enzymes with potential value as future therapeutic agents.

One of the unique and inventive technical features of the present invention is the use of XNAs to create analogues of the 10-23 DNAzyme. Without wishing to limit the invention to any theory or mechanism, it is believed that the technical feature of the present invention advantageously provides for an increased substrate binding kinetics without sacrificing multiple turnover activity, an improved cofactor binding, and a minimized the exolytic activity of biological enzymes. None of the presently known prior references or work have both unique inventive technical features of the present invention.

Furthermore, the prior references teach away from the present invention. For example, the present invention allows for multiple turnover activity that allows for robust sequence-specific gene silencing in mammalian cell culture.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the invention are apparent in the following detailed description and

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A-1D show the kinetic analysis of F10-23. FIG. 1A shows DNAzyme 10-23 (i.e., DNA arm (GATTGGAGCAACATCGATCGGAGTACT: SEQ ID NO: 67) in complex with an RNA substrate (CUAACCGUCAUGA: SEQ ID NO: 66), and the chemical structure of DNA. FIG. 1B shows F10-23 enzyme (i.e., FANA arm (GAUUGGAGCAACATCGATCGGAGUACU; SEQ ID NO: 68) in complex with an RNA substrate, and the chemical structure of FANA (bolded letter indicate FANA residues). FIGS. 1C-1D show the pre-steady state kinetic analysis of RNA cleavage by 10-23 and F10-23. Reactions were performed in a buffer containing either 10 MgCl₂ (FIG. 1C) or 1 MgCl₂ (FIG. 1D) and 150 mM NaCl at 24° C. (pH 7.5) with 0.5 μM substrate and 2.5 μM enzyme. Data points indicate percent substrate cleavage. Error bars denote ±standard deviation of the mean for n=3 independent replicates. FIGS. 1E-1F show single-turnover kinetic analysis of 10-23 and F10-23. FIG. 1E shows representative gels showing RNA cleavage activity in the presence of 10 mM MgCl₂. FIG. 1F shows representative gels showing RNA cleavage activity in the presence of 1 mM MgCl₂. All reactions were performed in a buffer containing 50 mM Tris-HCl (pH 7.5), and 150 mM NaCl at 24° C. S: full-length substrate, P: 5′ cleavage product. Molecular weight markers are indicated to the right of the gel.

FIGS. 2A-2F shows the engineering of the X10-23 nucleic acid enzyme. FIG. 2A shows the structure-activity mapping of the catalytic core of DNAzyme 10-23 by FANA substitutions. Data points indicate the relative substrate cleavage normalized to the wild-type 10-23 DNAzyme. Error bars denote±standard deviation of the mean for n=3 independent replicates. FIG. 2B shows the X10-23 enzyme (T*T*GAUUGGAGCAACAUCGATCGGAGUACUT*T*; SEQ ID NO: 69) in complex with an RNA substrate (CUAACCGUCAUGA: SEQ ID NO: 66). Legend: RNA (i.e., RNA substrate; top strand), DNA (black), FANA (i.e, FANA arm; underlined), and TNA (asterisk). FIGS. 2C-2D show the pre-steady state kinetic analysis of RNA cleavage by F10-23 and X10-23. Reactions were performed in a buffer containing either 10 MgCl₂ (FIG. 2C) or 1 MgCl₂ (FIG. 2D) and 150 mM NaCl at 24° C. (pH 7.5) with 0.5 μM substrate and 2.5 μM enzyme. Data points indicate percent substrate cleavage. Error bars denote±standard deviation of the mean for n=3 independent replicates. FIGS. 2E-2F show representative PAGE gels showing RNA cleavage by the engineered 10-23 variants in a buffer containing either 10 MgCl₂ (FIG. 2E) or 1 MgCl₂ (FIG. 2F). S: full-length substrate, P: 5′ cleavage product. Molecular weight markers are indicated to the right of the gel.

FIGS. 3A-3C shows 10-23 and F10-23 mediated cleavage of a long RNA substrate. FIG. 3A shows a schematic representation of 10-23 (left panel) and F10-23 (right panel) bound to a 103 nt mRNA transcript of human ribosomal modification protein rimK like family member A (RIMKLA). FIG. 3B shows representative gels showing RNA-cleavage reactions under single-turnover conditions. FIG. 3C shows representative gels showing RNA-cleavage reactions under stoichiometric and multiple-turnover reactions. All reactions were performed at 24° C. in a buffer containing 50 mM Tris-HCl (pH 7.5), 1 mM MgCl₂ and 150 mM NaCl. S: full-length substrate, 3′ P: 3′ cleavage product, 5′ P: 5′ cleavage product. Molecular weight markers are indicated to the left of the gel.

FIGS. 4A and 4B show the structure-activity mapping of the catalytic core of 10-23. FIGS. 4A and 4B show representative gels showing RNA-cleavage activity of single-point FANA substitutions (FIG. 4A) and combinations of multiple FANA substitutions (FIG. 4B). All reactions were performed under single-turnover conditions in 50 mM Tris-HCl (pH 7.5) containing 1 mM MgCl₂, 150 mM NaCl, at 24° C. “−” and “+” denote cleavage reactions quenched at reaction time point 0 and 40 min, respectively. M: cleavage reaction of 10-23 WT quenched after 40 min of reaction. S: full-length substrate, P: 5′ cleavage product. Molecular weight markers are indicated to the right of the gel

FIGS. 5A and 5B show the functional activity and biostability of X10-23. FIG. 5A shows representative PAGE gels showing RNA cleavage activity under steady-state and multiple-turnover conditions. RNA cleavage reactions were performed in buffer containing 50 mM Tris-HCl (PH 7.5), 1 mM MgCl₂ and 150 mM NaCl at 24° C. with 0.5 μM substrate and either 0.5 μM (steady-state) or 50 nM (multiple-turnover) enzyme. S: full-length substrate, P: 5′ cleavage product. FIG. 5B shows time-dependent biostability assay evaluated by denaturing PAGE. RNA cleaving enzymes were evaluated in DMEM containing 1 μM enzyme in the presence of 2 mg/mL of human liver microsome, 50% human serum (v/v), or 10 mU/mL of snake venom phosphodiesterase at 37° C. Molecular weight markers are indicated to the right of the gel.

FIGS. 6A-6D show the kinetic analysis of longer binding arms (7+7). FIG. 6A shows a schematic of F10-23 V2 (CUCUCUAGCAACATCGATCGGACCACG; SEQ ID NO: 71) in complex with its corresponding RNA substrate (GAGAGAGGUGGGUGC; SEQ ID NO: 70). Legend: FANA residues are underlined. FIG. 6B shows representative gel images showing the single-turnover RNA-cleavage activity of F10-23 V2 (top panel) and 10-23 V2 (bottom panel). Molecular weight markers indicated to the right of the gel. FIG. 6C shows pre-steady state kinetic plots of single-turnover cleavage reactions. Data points indicate percent substrate cleavage. Error bars denote±standard deviation of the mean for n=3 independent replicates. FIG. 6D shows representative gels showing RNA cleavage activity under stoichiometric and multiple-turnover enzyme-substrate conditions after different reaction times, as assayed by denaturing PAGE. Molecular weight markers indicated to the right of the gel. All reactions were performed in 50 mM Tris-HCl (pH 7.5) containing 1 mM MgCl₂, 150 mM NaCl, at 24° C. S: full-length substrate, P: 5′ cleavage product.

FIG. 7 shows the biostability of DNAzyme 10-23 in the absence and presence of an 3′ inverted dT nucleotide cap. Time-dependent biostability assay evaluated by denaturing PAGE. RNA-cleaving DNAzyme 10-23 was evaluated in DMEM containing 1 μM enzyme in the presence of 2 mg/mL of human liver microsome and 50% human serum (v/v) at 37° C. These assays show that the 3′ inverted dT cap protects the DNA enzyme from enzymatic degradation by 3′ exonucleases. The 3′ protected 10-23 DNAzyme was used as a control reagent in each of the cellular assays. Molecular weight markers are indicated to the right of the gel.

ME10-23
(UCAUGAGGCTAGCUACAACGAGGUUAG; SEQ ID NO: 52)
and
LNA10-23
(TCATGAGGCTAGCTACAACGAGGTTAG; SEQ ID NO: 53)

FIGS. 8A-8D show alternate 10-23 designs. FIGS. 8A and 8B show OME10-23 (FIG. 8A; (UCAUGAGGCTAGCUACAACGAGGUUAG; SEQ ID NO: 52)) and LNA10-23 (FIG. 8B; (TCATGAGGCTAGCTACAACGAGGTTAG; SEQ ID NO: 53)) in complex with an RNA substrate (CUAACCGUCAUGA: SEQ ID NO: 66). Legend: RNA (i.e., RNA substrate; top strand), DNA (black), OME (FIG. 8A, underlined), and LNA (FIG. 8B, underlined). FIG. 8C shows representative PAGE gels showing RNA cleavage activity under steady-state and multiple-turnover conditions. RNA cleavage reactions were performed in buffer containing 50 mM Tris-HCl (pH 7.5), 1 mM MgCl₂ and 150 mM NaCl at 24° C. with 0.5 UM substrate and either 0.5 μM (steady-state) or 50 nM (multiple-turnover) enzyme. S: full-length substrate, P: 5′ cleavage product. Molecular weight markers indicated to the right of the gel. FIG. 8D shows a structure-activity map of 10-23 activity for different chemical substitutions. Data points indicate percent substrate cleavage. Error bars denote±standard deviation of the mean for n=3 independent replicates.

FIGS. 9A-9D show kinetic analysis of OME10-23 and LNA10-23. FIGS. 9A and 9C show a pre-steady state kinetic analysis of RNA cleavage by OME10-23 and LNA10-23 enzymes in a buffer containing either 10 MgCl₂ (FIG. 9A) or 1 MgCl₂ (FIG. 9C) with 150 mM NaCl at 24° C. (pH 7.5). Data points indicate percent substrate cleavage. Error bars denote±standard deviation of the mean for n=3 independent replicates. FIGS. 9B and 9D show representative gels showing RNA cleavage activity in the presence of 10 mM MgCl₂ (FIG. 9B) and 1 mM MgCl₂ (FIG. 9D). All reactions were performed in a buffer containing 50 mM Tris-HCl (pH 7.5), and 150 mM NaCl at 24° C. S: full-length substrate, P: 5′ cleavage product. Molecular weight markers are indicated to the right of the gel.

FIGS. 10A-10C show GFP inhibition activity of X10-23 in HEK293 cells. FIG. 10A shows a schematic representation of X10-23 molecules used for the intracellular inhibition of GFP. HEK293 cells were co-transfected by X10-23 molecules targeting two different sites in the GFP mRNA transcript. Gene silencing activity levels were measured by GFP fluorescent cell imaging and qRT-PCR. FIG. 10B shows GFP fluorescent cell images collected prior to harvesting cells at 24 h post transfection. Scale bar, 100 μm. FIG. 10C shows a qRT-PCR analysis of DNA-free total RNA isolated 24 h post transfection using GFP-specific and GAPDH loading control primers. Two biological replicates and 3 technical replicates were collected for each condition, with one representative biological replicate shown in FIG. 10B and FIG. 10C. In FIG. 10C the error bars are presented as ±standard deviation of the mean for n=3 technical replicates.

FIGS. 11A and 11B show an in vitro validation of X10-23 constructs targeting GFP. FIG. 11A shows mRNA sequences (GGCAGCGUGCAGC: SEQ ID NO: 72 & UCUAUAGUGUCAC: SEQ ID NO: 73) targeted by X10-23. “GU” designated with an asterisk denotes the cleavage junction down-steam of the unpaired G. FIG. 11B shows a representative denaturing PAGE image showing the single-turnover mRNA cleavage activity of X10-23 on each target site. Reactions were performed under single-turnover conditions in a buffer containing 50 mM Tris-HCl (pH 7.5), 1 mM MgCl₂ and 150 mM NaCl at 24° C. S: full-length substrate, P: 5′ cleavage product. Molecular weight markers are indicated to the right of the gel.

FIG. 12 shows a dose-dependent actinomycin D analysis. HEK293T cells were transfected with 1 μg GFP target plasmid and 4 μg internal GFP X10-23 for 24 h. Twenty hours post-transfection, polymerase inhibitor, actinomycin D, was added to the transfected cultured cells at final concentrations of 0, 10, 20, or 40 μM and incubated for 4 h. Treated cells were harvested at 24 h post transfection. The data are presented with error bars as ±standard deviation of the mean from n=3 technical replicates.

FIGS. 13A and 13B show gene silencing comparison in HEK293T cells. HEK293T cells transfected with 1 μg GFP target plasmid were treated by co-transfection with dual X10-23 reagents (4 μg internal/4 μg 3′ UTR, XNA), dual DNAzyme 10-23 with 3′ inverted dT (DNA), dual inactive versions of X10-23 reagents, and dual FANA antisense strand capped at both ends with unpaired tT residues. At 44 h post transfection, 40 μM actinomycin D was added to transfected cultured cells for 4 h treatment. At 48 h incubation post transfection, cells were imaged then subjected to RNA isolation. FIG. 13A shows qRT-PCR analysis of DNA-free total RNA and FIG. 13B shows GFP fluorescent cell images. Two biological replicates and 3 technical replicates were collected for each condition, with one representative biological replicate shown. The data shown in FIG. 13A are presented with error bars±standard deviation of the mean from n=3 technical replicates. Scale bar denotes 100 μm.

FIG. 14A-14D shows the targeting of endogenous oncogene KRAS by x10-23 in cancer cells. FIG. 14A shows X10-23 target sites located at the first exon (AAACUUGUGGUAGU; SEQ ID NO: 74) and 3′UTR regions (ACAAUUUGUACUUUUU; SEQ ID NO: 75) of KRAS. The cleavage GU junctions are denoted by asterisks. FIG. 14B shows a schematic representation of the X10-23 molecule used for the inhibition of endogenous KRAS expressed by cancer cells. Cervical cancer cells (HeLa) and breast cancer cells (MDA-MB-231) were either transfected with individual X10-23 or with transfection carrier but no X10-23 (No XNA), and KRAS mRNA copy number was quantified by RT-qPCR. FIGS. 14C and 14D show an RT-qPCR analysis of DNA-free total RNA extracted from Hela cells (FIG. 14C) and MDA-MB-231 cells (FIG. 14D) 48 h post-transfection using KRAS-specific and GAPDH loading control primers. Two biological replicates and 3 technical replicates were collected for each of the cell lines, with one representative biological replicate shown. In FIGS. 14C and 14D the error bars are ±standard deviations of the mean for n=3 technical replicates.

FIGS. 15A and 15B show in vitro validation of X10-23 constructs targeting KRAS. FIG. 15A shows mRNA sequences targeted by X10-23 (first exon; UAAACUUGUGGUAG, SEQ ID NO: 76 & 3′UTR; ACAAUUUGUACUUUU, SEQ ID NO: 77). “GU” (denoted by asterisks) denotes the cleavage junction down-steam of the unpaired G. FIG. 15B shows a representative denaturing PAGE image showing the single-turnover mRNA cleavage activity of X10-23 on each target site. Reactions were performed under single-turnover conditions in a buffer containing 50 mM Tris-HCl (pH 7.5), 1 mM MgCl₂ and 150 mM NaCl at 24° C. S: full-length substrate, P: 5′ cleavage product. Molecular weight markers are indicated to the right of the gel.

FIG. 16A-16F shows the mechanistic analysis of X10-23. FIGS. 16A, 16B and 16C show representative gels showing RNA cleavage activity in the presence and absence of RNase H for an internal segment of GFP (GGCAGCGUGCAGC, SEQ ID NO: 72) and FIGS. 16D, 16E, and 16F shows the first exon segment (UAACUUGUGGUAG, SEQ ID NO: 78) of KRAS RNA. Legends: RNA (i.e., RNA substrate; top strand, DNA (black), FANA (underlined), and TNA (asteriks). FIGS. 16A and 16D show X10-23 with an active catalytic core (T*CCGUCGAGCAACAUCGATCGGACGUCGT *; SEQ ID NO: 79 (FIG. 16A) or T*AUUGAAAGCAACAUCGATCGGACCAUCT*; SEQ ID NO: 80 (FIG. 16D). FIGS. 16B and 16E show X10-23 with an inactive catalytic core (T*CCGUCGGGCTAGCTACAACGAACGUCGT*; SEQ ID NO: 81 (FIG. 16B) or T*AUUGAAGGCTAGCTACAACGAACCAUCT*; SEQ ID NO: 82 (FIG. 16E)). FIGS. 16C and 16F show X10-23 with an active catalytic core that does not hybridize to the RNA target (T*AGAUAUAGCAACAUCGATCGGACAGUGT*; SEQ ID NO: 83 (FIG. 16C) or T*UGUUAAAGCAACAUCGATCGG\sqrt{AUGAAAA}T*; SEQ ID NO: 84 (FIG. 16F)). All assays were performed in a buffer containing 0.5 mM MgCl₂ and 150 mM NaCl at 37° C. (pH 7.5) with 1 μM substrate and 1 μM enzyme. Nuclease reactions included 0.1 unit/uL of RNase H. S: full-length substrate, P: 5′ cleavage product. Molecular weight markers are indicated to the right of the gel.

FIG. 17 shows X10-23 KRAS gene silencing control in Hela cells. Hela cells were treated with 5.9 μg of active X10-23, inactive X10-23, and active X10-23 with non-complementary binding arms (unpaired) reagents targeting the 3′UTR region of KRAS for 96 h. cDNA from DNA-free total RNA of condition was subjected to qPCR analysis to assess KRAS mRNA copy number. Data are presented with error bars as ±standard deviation of the mean for n=3 technical replicates.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting. All embodiments disclosed herein can be combined with other embodiments unless the context clearly dictates otherwise.

### Terms

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

The term “disease” or “disorder” or “condition” refers to any alteration in state of the body or of some of the organs, interrupting or disturbing the performance of their functions and/or causing symptoms such as discomfort, dysfunction, distress, or even death to the person afflicted or those in contact with a person. A disease or disorder or condition can also be related to a distemper, ailing, ailment, malady, disorder, sickness, illness, complaint, indisposition or affliction.

As used herein, the terms “treat” or “treatment” or “treating” refers to both therapeutic treatment and prophylactic or preventative measures, wherein the object is to prevent or slow the development of the disease, such as slow down the development of a disorder, or reducing at least one adverse effect or symptom of a condition, disease or disorder, e.g., any disorder characterized by insufficient or undesired organ or tissue function. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects

As used herein, the term “XNA” or “xeno-nucleic acids” may refer to artificial genetic polymers with novel sugar-phosphate backbones that harbor unique physicochemical properties relative to natural deoxyribonucleic acid (DNA) and ribonucleic acid (RNA).

As used herein, the term “FANA” or “2′-fluoroarabino nucleic acid” refers to an artificial nucleic acid wherein the sugar portion of the nucleic acid is 2-fluoroarabinose.

As used herein, the term “TNA” or “α-L-threofuranosylnucleic acid” or “threose nucleic acid” refers to an artificial nucleic acid wherein the sugar portion of the nucleic acid is threose.

As used herein, the term “LNA” or “locked nucleic acids” may refer to modified RNA nucleotides in which the ribose moiety is modified with an extra bridge connecting the 2′ oxygen and 4′ carbon.

As used herein, the term “DNAzyme 10-23” refers to an enzyme comprising a 15-nucleotide (nt) catalytic domain (5′-GGCTAGCTACAACGA-3′ (SEQ ID NO: 1) that is flanked on both sides by substrate binding arms (i.e., substrate recognition domains) that can vary in length depending on the sequence of the RNA substrate, typically 6-20 nts. In some embodiments, the two substrate recognition domains are designed to achieve target specificity.

Target specificity is based on complementary Watson-Crick base pairing between the RNA target (i.e., a target selected by a user; e.g., KRAS RNA) and the substrate binding arms of the DNAzyme. In some embodiments, the DNAzyme cleaves a G-U dinucleotide junction; therefore the binding arms may be designed to be complementary to the RNA regions flanking the G-U cut site.

In some embodiments, the two substrate recognition domains recognize an RNA target through complementary Watson-Crick base pairing. Once the target RNA is bound by the substrate recognition domains, RNA cleavage ensues at a predefined purine-pyrimidine (R-Y) junction with the highest activity levels observed for G-U dinucleotides. In some embodiments, the cleavage mechanism involves metal-assisted deprotonation of a 2′-hydroxyl from the purine (R) nucleotide, followed by nucleophilic attack on the neighboring phosphodiester bond to yield an upstream cleavage product with a 2′,3′-cyclic phosphate and a downstream cleavage product with a 5′-hydroxyl group.

In some embodiments, the substrate recognition domains may range from 6-20 nucleotides long. In some embodiments, the substrate recognition domains are at least 4 nucleotides long. In some embodiments, the substrate recognition domains are at least 5 nucleotides long. In some embodiments, the substrate recognition domains are at least 6 nucleotides long. In some embodiments, the substrate recognition domains are at least 7 nucleotides long. In some embodiments, the substrate recognition domains are at least 8 nucleotides long. In some embodiments, the substrate recognition domains are at least 9 nucleotides long. In some embodiments, the substrate recognition domains are at least 10 nucleotides long. In some embodiments, the substrate recognition domains are at least 12 nucleotides long. In some embodiments, the substrate recognition domains are at least 14 nucleotides long. In some embodiments, the substrate recognition domains are at least 16 nucleotides long. In some embodiments, the substrate recognition domains are at least 18 nucleotides long. In some embodiments, the substrate recognition domains are at least 20 nucleotides long. In some embodiments, the substrate recognition domains are at least 22 nucleotides long.

As used herein “X10-23” and “XNAzyme” may be used interchangeably.

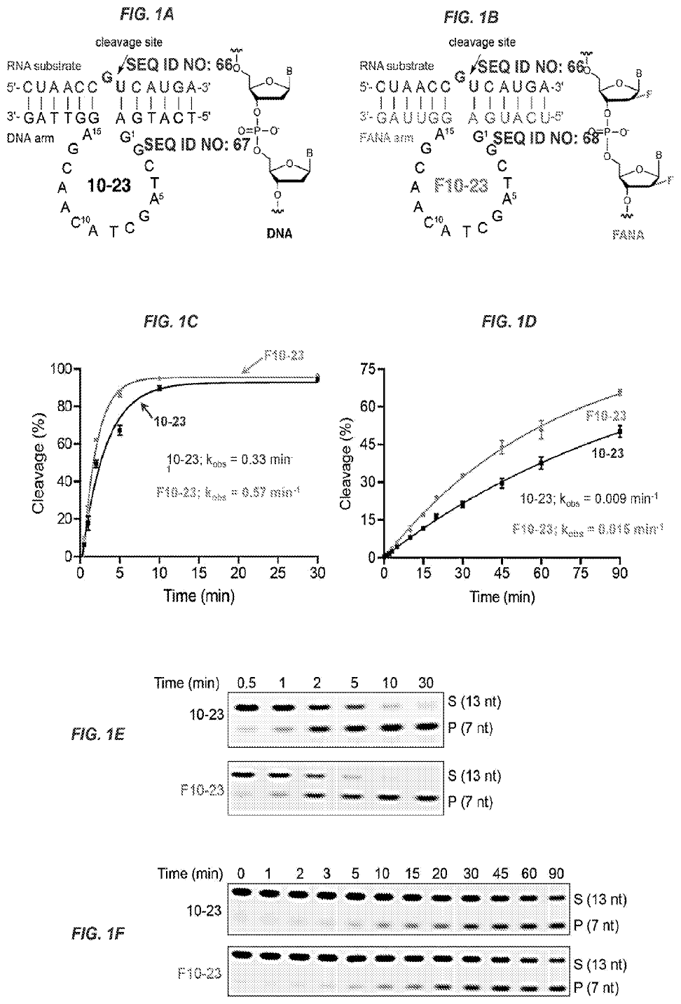

Referring now to FIGS. 1A-17, the present invention features a DNAzyme 10-23 analog compositions (X10-23) that allow for an increased substrate binding kinetics without sacrificing multiple turnover activity, an improved cofactor binding, and minimize the exolytic activity of biological enzymes. X10-23 composition described herein features 2′-fluoroarabino nucleic acid (FANA) and threose nucleic acid (TNA) residues at specific locations. The present invention also features methods of use for the X10-23 composition described herein.

The present invention features a composition for gene silencing. In some embodiments, the composition comprises a 15-nucleotide catalytic domain according to SEQ ID NO: 1, a first substrate recognition domain 5′ to the catalytic domain, a second substrate recognition domain 3′ to the catalytic domain, a 5′ terminal threose nucleic acid (TNA) residue and a 3′ terminal TNA residue. In some embodiments, the composition comprises a catalytic domain wherein one or more nucleic acids of the catalytic domain is replaced by xeno-nucleic acids (XNA). In other embodiments, one or more nucleic acids of the first substrate recognition domain or the second recognition domain is replaced by XNA. In other embodiments, the composition has enhanced stability and enhanced catalytic activity compared to a control molecule comprising wild type SEQ ID NO: 1 as its catalytic domain.

The XNAzymes herein comprise one or more alternative nucleic acid residues in the 15-residue catalytic core. Additionally, the XNAzymes comprise two substrate binding arms flanking the catalytic domain that in some embodiments are composed entirely of alternative nucleic acid residues. Examples of alternative nucleic acid residues include 2′-fluoroarabino nucleic acid (FANA) and threose nucleic acid (TNA). The present invention is not limited to TNA and FANA. Other XNA examples include but are not limited to: hexose nucleic acid (HNA), cyclohexenyl nucleic acid (CeNA), glycerol nucleic acid (GNA), peptide nucleic acid (PNA), arabino nucleic acid (ANA), phosphonomethyl-threosyl nucleic acid (tPhoNA), locked nucleic acid (LNA), pyranosyl-RNA (pRNA), xylo nucleic acid (XNA), and deoxy-xylonucleic acid (dXNA).

In some embodiments, the XNA is 2′-fluoroarabino nucleic acid (FANA). In other embodiments, the XNA is threose nucleic acid (TNA).

The present invention features a composition for gene silencing, the composition comprising an 10-23 analogue, wherein one or more sugars of the nucleotides in the 10-23 analogue is replaced by threose or 2′-fluoroarabinose.

As a non-limiting example, the XNAzyme may comprise FANA substitutions in the substrate recognition domains (substrate binding arms). The XNAzyme may further comprise TNA residues flanking the ends of the FANA substrate recognition domains. The XNAzyme may further comprise TNA substitutions, e.g., in the catalytic core.

TABLE 1
Examples of XNAzymes Catalytic Domain Sequences

| | Sequence of Catalytic Domain (5′-3′) | | | | | | | | | | | | | | |
|---|---|---|---|---|---|---|---|---|---|---|---|---|---|---|---|
| SEQ. ID | (residue number) | | | | | | | | | | | | | | |
| NO: | 1 | 2 | 3 | 4 | 5 | 6 | 7 | 8 | 9 | 10 | 11 | 12 | 13 | 14 | 15 |
| 1 | G | G | C | T | A | G | C | T | A | C | A | A | C | G | A |
| 2 | fG | G | C | T | A | G | C | T | A | C | A | A | C | G | A |
| 3 | G | fG | C | T | A | G | C | T | A | C | A | A | C | G | A |
| 4 | G | G | fC | T | A | G | C | T | A | C | A | A | C | G | A |
| 5 | G | G | C | fT | A | G | C | T | A | C | A | A | C | G | A |
| 6 | G | G | C | T | fA | G | C | T | A | C | A | A | C | G | A |
| 7 | G | G | C | T | A | fG | C | T | A | C | A | A | C | G | A |
| 8 | G | G | C | T | A | G | fC | T | A | C | A | A | C | G | A |
| 9 | G | G | C | T | A | G | C | fT | A | C | A | A | C | G | A |
| 10 | G | G | C | T | A | G | C | T | fA | C | A | A | C | G | A |
| 11 | G | G | C | T | A | G | C | T | A | fC | A | A | C | G | A |
| 12 | G | G | C | T | A | G | C | T | A | C | fA | A | C | G | A |
| 13 | G | G | C | T | A | G | C | T | A | C | A | fA | C | G | A |
| 14 | G | G | C | T | A | G | C | T | A | C | A | A | fC | G | A |
| 15 | G | G | C | T | A | G | C | T | A | C | A | A | C | fG | A |
| 16 | G | G | C | T | A | G | C | T | A | C | A | A | C | G | fA |
| 17 | G | fG | C | T | A | G | C | fT | A | C | A | A | C | G | A |
| 18 | G | fG | C | fT | A | G | C | fT | fA | C | A | A | C | G | A |

(f = FANA residue;
t = TNA residue)

In some embodiments, the 10-23 analogue (X10-23) silences genes through knocking down a target RNA.

The present invention may also feature a method of treating a disease or condition or a symptom thereof. In some embodiments, the method comprises administering an effective amount of a 10-23 analogue composition to a subject in need thereof. In some embodiments, the composition comprises a 15-nucleotide catalytic domain according to SEQ ID NO: 1, a first substrate recognition domain 5′ to the catalytic domain, a second substrate recognition domain 3′ to the catalytic domain, a 5′ terminal threose nucleic acid (TNA) residue and a 3′ terminal TNA residue. In some embodiments, the composition comprises a catalytic domain wherein one or more nucleic acids of the catalytic domain is replaced by xeno-nucleic acids (XNA). In other embodiments, one or more nucleic acids of the first substrate recognition domain or the second recognition domain are replaced by XNA.

In some embodiments, the target RNA is KRAS. Other target RNAs may be used in accordance with compositions and methods as described herein.

In some embodiments, one X10-23 composition may target a single RNA (i.e., a single target RNA). In some embodiments, one or more X10-23 compositions may target a single RNA (i.e., a single target RNA). In some embodiments, a X10-23 composition may be designed to target any purine-pyrimidine dinucleotide junction (R-Y) of a target RNA. In some embodiments, a purine-pyrimidine dinucleotide junction (R-Y) of R-uracil (R-U) is preferred over R-cysteine (R-C). In preferred embodiments, the X10-23 composition described herein targets a purine-uracil (R-U) dinucleotide junction. In other embodiments, the X10-23 composition described herein targets a purine-cysteine (R-C) dinucleotide junction.

The present invention features a method of validating and treating a disease or condition, or a symptom thereof caused by a genetic mutation in the mRNA strand, the method comprising administering an effective amount of a 10-23 analogue to a subject in need thereof.

In some embodiments, the disease or condition is caused by a common or rare genetic disease, viral or bacterial pathogen, cancer, inflammation, cardiovascular disease, immune deficiency or a neurological disorder. In other embodiments, the disease or condition is pancreatic, and colorectal adenocarcinomas.

TABLE 2
Table of oligonucleotides used for in vitro testing

| | | |
|---|---|---|
| | | SEQ |
| Oligo Name | Sequence (5′-3′) | ID NO: |

Oligonucleotides for F10-23 (FANA substitution in binding arms)

| | | |
|---|---|---|
| F10-23 | ucaugaGGCTAGCTACAACGAgguuag | 19 |
| (6 nt binding arms) | | |
| 10-23 | TCATGAGGCTAGCTACAACGAGGTTAG | 20 |
| (6 nt binding arms) | | |
| 10-23 | GTCATGAGGCTAGCTACAACGAGGTTAGG | 21 |
| (7 nt binding arms) | | |
| RNA substrate | /5Cy5/rCrUrArArCrCrGrUrCrArUrGrA | 22 |
| (6 nt binding arms) | | |
| RNA substrate | /5Cy5/rCrCrUrArArCrCrGrUrCrArUrGr | 23 |
| (7 nt binding arms) | ArC | |
| F10-23 V2 | gcacccaGGCTAGCTACAACGAcucucuc | 24 |
| (7 nt binding arms) | | |
| 10-23 V2 | GCACCCAGGCTAGCTACAACGACTCTCTC | 25 |
| (7 nt binding arms) | | |
| RNA substrate V2 | /5Cy5/rGrArGrArGrArGrGrUrGrGrGrUr | 26 |
| (7 nt binding arms) | GrC | |
| 103 nt RNA | rCrCrUrUrUrUrCrUrUrArArUrUrUrGrArUr | 27 |
| containing target site | UrCrCrUrGrCrCrArArCrArCrCrCrUrArArC | |
| | rCrGrUrCrArUrGrArCrUrUrCrArCrCrUrUr | |
| | UrUrArCrCrArUrGrGrGrUrGrGrCrUrUrArU | |
| | rUrArArArArArCrArUrUrUrUrCrCrUrGrGr | |
| | ArCrGrArArGrArCrArGrUrUrCrCrCrU | |

Oligonucleotides for mapping FANA
substitutions in the catalytic core

| | | |
|---|---|---|
| 10-23 G1 | TCATGAgGCTAGCTACAACGAGGTTAG | 28 |
| 10-23 G2 | TCATGAGgCTAGCTACAACGAGGTTAG | 29 |
| 10-23 C3 | TCATGAGGcTAGCTACAACGAGGTTAG | 30 |
| 10-23 U4 | TCATGAGGCuAGCTACAACGAGGTTAG | 31 |
| 10-23 A5 | TCATGAGGCTaGCTACAACGAGGTTAG | 32 |
| 10-23 G6 | TCATGAGGCTAgCTACAACGAGGTTAG | 33 |
| 10-23 C7 | TCATGAGGCTAGcTACAACGAGGTTAG | 34 |
| 10-23 U8 | TCATGAGGCTAGCuACAACGAGGTTAG | 35 |
| 10-23 A9 | TCATGAGGCTAGCTaCAACGAGGTTAG | 36 |
| 10-23 C10 | TCATGAGGCTAGCTACAACGAGGTTAG | 37 |
| 10-23 A11 | TCATGAGGCTAGCTACaACGAGGTTAG | 38 |
| 10-23 A12 | TCATGAGGCTAGCTACAaCGAGGTTAG | 39 |
| 10-23 C13 | TCATGAGGCTAGCTACAAcGAGGTTAG | 40 |
| 10-23 G14 | TCATGAGGCTAGCTACAACgAGGTTAG | 41 |
| 10-23 A15 | TCATGAGGCTAGCTACAACGaGGTTAG | 42 |
| 10-23 G2/U8 | TCATGAGgCTAGCuACAACGAGGTTAG | 43 |
| 10-23 G2/U4/U8/A9 | TCATGAGgCuAGCuaCAACGAGGTTAG | 44 |

Oligonucleotides for biostability test

| | | |
|---|---|---|
| 10-23 WT | TCATGAGGCTAGCTACAACGAGGTTAG | 20 |
| F10-23 | ucaugaGGCTAGCTACAACGAgguuag | 19 |
| X10-23 | ^{t}TucaugaGgCTAGCuACAACGAgguuag^{t}T | 45 |

Oligonucleotides for GFP reductions

| | | |
|---|---|---|
| X10-23 targeting GFP | ^{t}TgcugcaGgCTAGCuACAACGAgcugcc^{t}T | 46 |
| internal | | |
| X10-23 targeting GFP | ^{t}TgugacaGgCTAGCuACAACGAuauaga^{t}T | 47 |
| 3′UTR | | |
| 10-23 targeting GFP | GCTGCAGGCTAGCTACAACGAGCTGCC/3InvdT/ | 48 |
| internal | | |
| 10-23 targeting GFP | GTGACAGGCTAGCTACAACGATATAGA/3InvdT/ | 49 |
| 3′ UTR | | |
| X-antisense targeting | ^{t}Tgcugcacgcugcc^{t}T | 50 |
| GFP internal | | |
| X-antisense targeting | ^{t}Tgugacacuauaga^{t}T | 51 |
| GFP 3′ UTR | | |

Letters in bold lower cases denote FANA nucleotides; RNA residues are denoted by “r”; TNA residues are denoted by superscript “t”; “3InvdT” denotes 3′ inverted dT; “m” denotes 2′-O-methyl ribonucleotides; “+” denotes LNA residues.

The present invention may further feature a method of validating gene mutations associated with a disease or condition. In some embodiments, the method comprises administering a 10-23 analogue composition to a cell line or animal model, and analyzing the cell line or animal model for characteristics associated with the disease or condition. In some embodiments, the composition comprises a 15-nucleotide catalytic domain according to SEQ ID NO: 1 with one or more nucleic acids of the catalytic domain are replaced by xeno-nucleic acids (XNA), a first substrate recognition domain 5′ to the catalytic domain, a second substrate recognition domain 3′ to the catalytic domain, a 5′ terminal threose nucleic acid (TNA) residue, and a 3′ terminal TNA residue. In some embodiments, one or more nucleic acids of the first substrate recognition domain or the second recognition domain are replaced by XNA.

As used herein, an appropriate cell line refers to a cell line that is biologically relevant to the disease or the condition being studied. In some embodiments, cell lines may include, but are not limited to, HEK-293, HeLa, or Chinese hamster ovary cells (CHO). As used herein, “characteristics associated with a disease or condition” may refer to measurable molecular changes in a cell line.

### EXAMPLE

The following is a non-limiting example of the present invention. It is to be understood that said example is not intended to limit the present invention in any way. Equivalents or substitutes are within the scope of the present invention.

### Optimizing the Substrate Recognition Domain.

XNA containing oligonucleotide synthesis and preparation: TNA phosphoramidites were synthesized using known methods. Standard β-cyanoethyl phosphoramidite chemistry and an Applied Biosystems 3400 DNA Synthesizer were used to synthesize TNA oligonucleotides on Universal Support II CPG columns in 1 μmole scale. Standard DNA coupling procedures were modified for FANA and/or TNA containing oligonucleotides such that coupling time for FANA and TNA amidites was increased to 360 s and 1200 s, respectively. Detritylation was performed in two cycles for TNA amidites, 60 s each. Oligonucleotides used for biostability studies were coupled with a 5′-hexynyl phosphoramidite for later IR-680 fluorophore tagging via click chemistry. Cleavage from the solid support and final deprotection of oligonucleotides synthesized were achieved simultaneously in NH₄OH (33%) for 18 h at 55° C. Oligonucleotides were purified on denaturing (8 M urea) PAGE, recovered by electro-elution, and subsequently desalted by buffer exchange using microcentrifugal concentrators, and quantified by nano-drop. All oligonucleotides synthesized in-house were subjected to Quadrupole time-of-flight mass spectrometry (Q-TOF) for identity confirmation.

Recognizing that XNAs harbor physicochemical properties that are distinct from those found in natural DNA and RNA, whether XNA residues could be used to enhance the RNA-cleavage activity of 10-23 under physiological conditions needed to first be determined. First, all of the DNA residues were replaced in the binding arms of the substrate recognition domain with 2′-fluoroarabino nucleic acids (FANA, FIG. 1B). FANA is a close structural analog of DNA that contains a fluorine atom at the 2′ position of a 2′-deoxyarabinose sugar.

Apart from thymidine, which was substituted for uridine, each DNA nucleotide was replaced with the corresponding FANA nucleotide. LNA was also considered as a possible XNA modification due to its high thermal stability with RNA (˜2-3° C. per base pair), but concerns over its cellular toxicity led to focusing the efforts on FANA. This version of 10-23, termed F10-23, functions with a pseudo first-order rate constant (k_{obs}) of 0.57 min-1 under single-turnover conditions in the buffer containing 10 mM MgCl₂ and 150 mM NaCl (pH 7.5, 24° C.), which is nearly 2-fold faster than the unmodified parent enzyme (FIG. 1C, FIG. 1E). The enhanced activity of F10-23 over 10-23 is maintained under physiological conditions in which the Mg²⁺ concentration is reduced to 1 mM (k_{obs} of 0.015 min-1 versus 0.009 min-1, respectively) (FIG. 1D, FIG. 1F)

### Cleavage of Long RNA Substrates:

Generation of RIMKLA mRNA transcript for in vitro cleavage assay by 10-23 and F10-23: Homo sapiens ribosomal modification protein rimK like family member A (RIMKLA) was reversed transcribed from HeLa total RNA using SuperScript RT III (Invitrogen-Life Technologies, CA) according to manual instruction. RIMKLA cDNA was subjected to 2-round nested PCR using KOD polymerase (Fisher Scientific, Cat #710863) to introduce T7 promoter upstream of the coding sequence of RIMKLA for subsequent in vitro transcription. mRNA was then transcribed in 1×RNAPol reaction buffer, supplemented with 0.5 mM each ATP, UTP, GTP, CTP, 5 mM dithiothreitol (DTT), 1 U/μL RNase inhibitor using 4 μg/μL of the purified DNA amplicon and 25 U/μL of T7 RNA polymerase at 37° C. for 16 h. Transcription was terminated by the addition of 10 U/mL of RNase-free DNase I and incubation at 37° C. for 15 min. The transcription reaction was resolved by 10% denaturing purification PAGE (8 M urea), and the gel was visualized by UV-shadowing. The RNA transcript was excised, electroeluted, exchanged into H2O using EMD Millipore YM-3 micro centrifugal device, and UV quantified by Nanodrop before in vitro kinetic cleavage assays by 10-23 and F10-23.

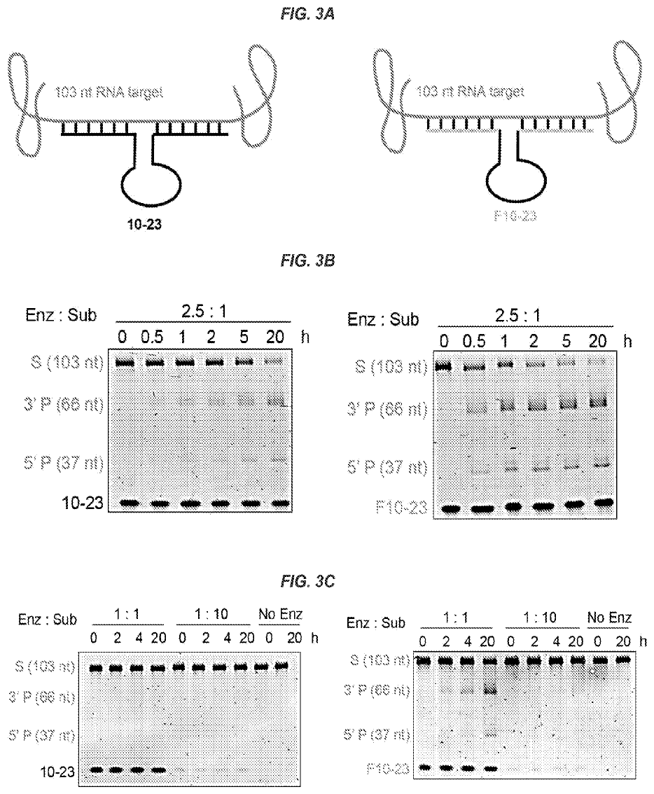

To provide further evidence of RNA cleavage activity, F10-23 was challenged to cut a much longer RNA substrate that was more reminiscent of a biological RNA molecule found in nature. For this example, a 103 nt segment of the ribosomal modification protein rimK, was chosen, and an unlabeled RNA transcript was generated by in vitro transcription with T7 RNA polymerase. The catalytic activity of F10-23 was compared to standard 10-23 under single-turnover, steady-state, and multiple-turnover conditions in a physiological buffer containing 1 mM MgCl₂ and 150 mM NaCl (pH 7.5, 24° C.). Here steady-state kinetic measurements are viewed as a more rigorous test of catalytic activity than the more common single-turnover reaction. Analysis of the reaction products by denaturing polyacrylamide gel electrophoresis (PAGE) (FIGS. 3A and 3B) with ethidium bromide staining reveals the appearance of upstream (37 nt) and downstream (66 nt) cleavage fragments produced by sequence-specific cleavage of a central G-U dinucleotide junction. F10-23 is faster on the shorter RNA substrate than the parent 10-23 enzyme in all cases tested, suggesting that F10-23 has the potential to invade folded RNA structures found in cellular systems.

### Optimizing the Catalytic Core:

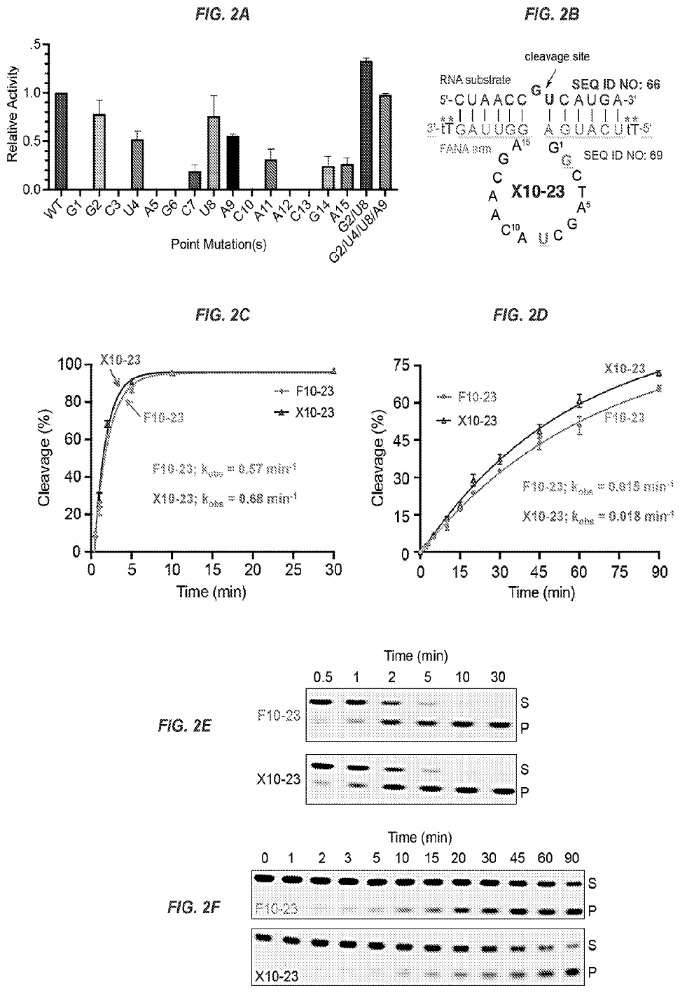

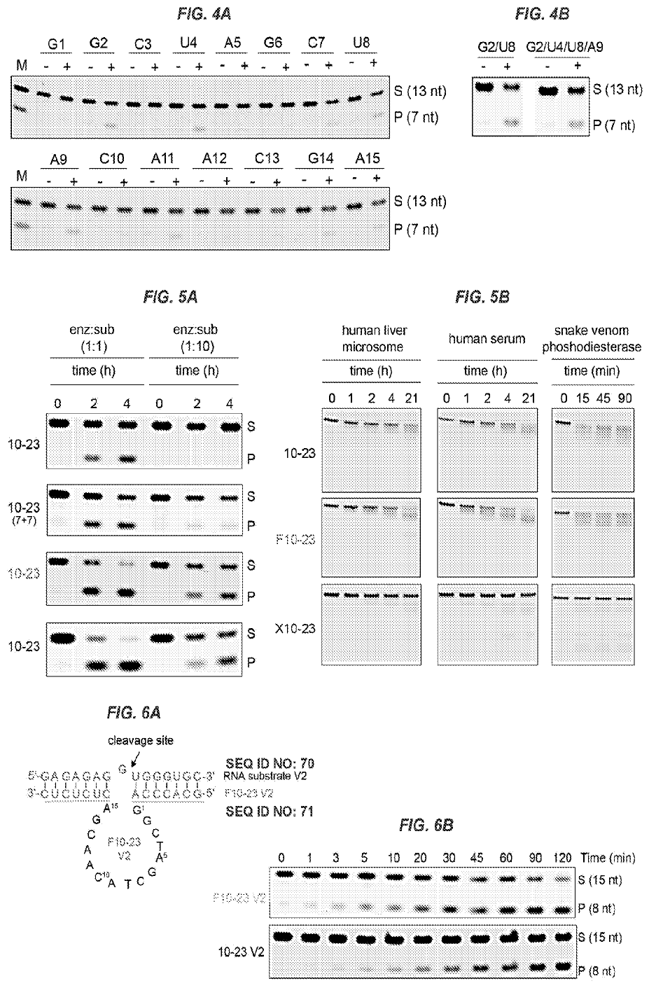

To determine if the activity of 10-23 could be further enhanced by introducing chemical modifications into the catalytic domain. Although this region of the sequence represents an evolutionary optimum in which almost any nucleotide change leads to a mutant enzyme with reduced catalytic activity, substantially less is known about the tolerance of the catalytic core toward chemical modifications that alter the sugar moiety. Each DNA residue was systematically replaced in the catalytic core with the corresponding FANA nucleotide (e.g., dA was replaced with fanaA (i.e, fA); Table 1). The complete set of 15 single-point mutant enzymes were assayed for RNA cleavage activity under single-turnover conditions in a physiological buffer containing 1 mM MgCl₂ and 150 mM NaCl (pH 7.5, 24° C.). The catalytic profile (FIG. 2A, FIG. 4A) indicates that residues G2 and T8 are highly tolerant to substitution with the FANA residues, maintaining ˜80% activity relative to the parent enzyme. This result shows that G2 and T8 accept conformational distortions caused by 2′-5′ phosphodiester linkages. Of the remaining positions, substitutions made to the T4 and A9 positions show moderate activity (50-60%), while the C9, G14, and A15 positions have low activity (20-30%). The remaining 8 positions are each inactive when the wild-type DNA residue is replaced with FANA. Surprisingly, a designed version of 10-23 carrying both the G2 and U8 FANA mutations is nearly 50% more active than the parental enzyme FIG. 4B), suggesting that these two substitutions function with synergistic activity.

### Designing the X10-23 Enzyme:

Encouraged by the structural mutagenesis study, a new version of the enzyme was synthesized in which the catalytic core of F10-23 was modified to contain both G2 and U8 FANA substitutions (SEQ ID NO: 17) and non-complementary α-L-threofuranosyl thymidine (tT) residues were added to the 5′ and 3′ terminal positions to protect the oligonucleotide against nuclease digestion (FIG. 2B). α-L-threofuranosyl nucleic acid (TNA) is an artificial genetic polymer in which the natural five-carbon ribose sugar found in RNA has been replaced with an unnatural four-carbon threose sugar. TNA is an ideal choice for stabilizing the backbone structure against nuclease digestion as TNA is completely recalcitrant to DNA and RNA degrading enzymes This new version of the enzyme, termed X10-23, contains three different classes of nucleic acids (DNA, FANA, and TNA) and functions with a pseudo first-order rate constants of 0.68 min-1 and 0.018 min-1 in reaction buffer [150 mM NaCl (pH 7.5, 24° C.)] containing 10 mM and 1 mM MgCl₂, respectively (FIGS. 2C, 2D, 2E, and 2F). These rates are at least 2-fold faster than the parent enzyme.

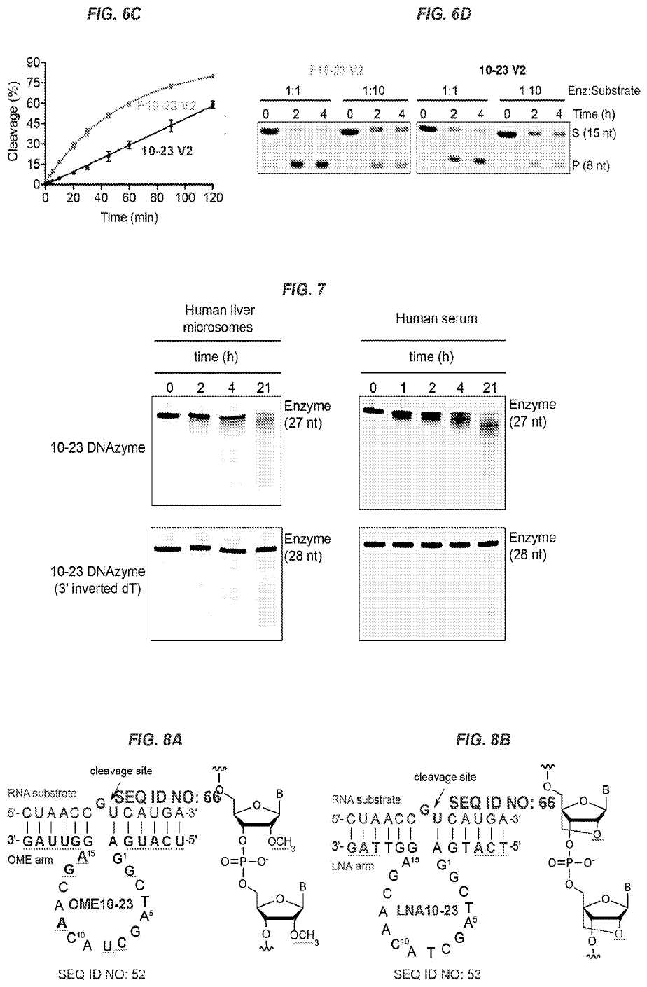

Next, whether the enhanced chemical diversity of X10-23 enabled higher multiple-turnover activity in vitro was determined. First 10-23, F10-23, and X10-23 were evaluated under steady-state conditions with equimolar concentrations of substrate and enzyme. Kinetic measurements reveal that F10-23 and X10-23 (FIG. 5A) are ˜3-fold faster than 10-23, which is consistent with their activity under pre-steady-state conditions. However, striking differences were observed under multiple turnover conditions when the RNA substrate is present in 10-fold molar excess over the enzyme. Under these conditions, F10-23 and X10-23 are ˜50-fold more active than the parent enzyme, suggesting that important structural differences exist between the pre- and post-catalytic state of X10-23 versus the parent 10-23 DNAzyme. No significant difference in catalytic activity was observed when the binding arms of the parent enzyme were extended by 1 nt (7+7 rather than 6+6) (FIG. 5A). Furthermore, product inhibition was not observed when the analogous 7+7 construct was tested for F10-23 (FIG. 6A-6D), indicating that FANA provides a balanced solution to the problem of how to enhance substrate binding kinetics while avoiding the harmful effects of product inhibition.

### Evaluating the Biostability of X10-23

Biostability measurements: All biostability assays were performed in DMEM containing 1 μM of tested construct with the presence of 2 mg/mL of human liver microsome, or 50% human serum (v/v), or 10 mU/mL of snake venom phosphodiesterase at 37° C. Multiple time points were collected for each condition by quenching 1.5 μL of reactions using 15 μL (10 equivalents, v/v) of formamide containing 25 mM EDTA. Samples were denatured for 15 min at 95° C. and analyzed by 15% denaturing PAGE. Gels were visualized using a LI-COR Odyssey CLx.

Along with efficient catalytic activity, biostability is a critical parameter for achieving improved efficacy in cellular systems that contain strong DNA and RNA degrading enzymes. For this assay, the stability of the 10-23, F10-23, and X10-23 scaffolds was analyzed in concentrated human liver microsomes (HLM) and 50% human serum (HS) in Dulbecco's Modified Eagle Medium (DMEM). Both assays provide a rigorous test of oligonucleotide stability due to the abundance and diversity of nucleases present in the media. In addition, each scaffold was also evaluated against snake venom phosphodiesterase (SVPDE), an aggressive enzyme with strong 3′-exonuclease activity commonly employed to evaluate the stability of oligonucleotide therapeutics. The results (FIG. 5B, FIG. 7) clearly show that X10-23 displays markedly enhanced biostability under all conditions tested, with almost no degradation observed after either 21 hours of incubation in HLM and HS or 90 minutes of incubation in SVPDE. By comparison, 10-23 and F10-23 show significant degradation with F10-23 being slightly less stable than 10-23. This result validates the utility of TNA as a capping agent for protecting the 5′ and 3′ termini against nuclease digestion.

### Comparing X23-10 to 2′-O-Methyl RNA and LNA Version of 10-23:

Kinetic cleavage reaction of 10-23, F10-23, X10-23, OME10-23, and LNA10-23: Single-turnover kinetic cleavage reactions were conducted in 50 mM Tris buffer (pH 7.5) containing 150 mM NaCl, 1 mM MgCl₂, 0.5 μM of substrate, and 2.5 μM of enzyme at 24° C. Purified enzymes and substrates were annealed in a 50 mM Tris buffer (pH 7.5) by heating for 5 min at 90° C. and cooling for 5 min on ice. Reactions were initiated by the addition of NaCl and MgCl₂ to the reaction. For determination of pseudo first-order rate constant, multiple time points were collected by quenching 1.5 L of reaction using 15 μL (10 equivalents, v/v) of formamide stop buffer (99% deionized formamide, 25 mM EDTA) and cooling on ice. Samples were denatured for 15 min at 95° C. and analyzed by 15% denaturing PAGE. Gels were visualized and quantified using a LI-COR Odyssey CLx. Values of k_{obs} were calculated by fitting the percentage of substrate cleaved and reaction time (min) to the first-order decay equation (1) using Prism 6 (GraphPad, USA):

$${P}_{t} = {P}_{∞} ( 1 - {e}^{- {k}_{obs} t} ) \qquad ( 1 )$$

where Pₜ is the percentage of cleaved substrate at time t, P_{∞} is the apparent reaction plateau and k_{obs} is the observed first-order rate constant. For kinetic cleavage reactions under stoichiometric and multiple-turnover conditions, substrate concentrations were poised at 0.5 μM, and enzyme concentrations were adjusted to 0.5 μM and 50 nM, respectively.

How X10-23 compared to other chemically enhanced versions of 10-23 that have been previously evaluated as gene silencing reagents was determined next. Among the various combinations, 2′-O-methyl ribonucleotides and LNA have received significant attention as chemical modifications that function with enhanced activity and biostability which is consistent with their broad deployment in other classes of therapeutic oligonucleotides.

OME10-23
(UCAUGAGGCTAGCUACAACGAGGUUAG; SEQ ID NO: 52)
and
LNA10-23
(TCATGAGGCTAGCTACAACGAGGTTAG; SEQ ID NO: 53),

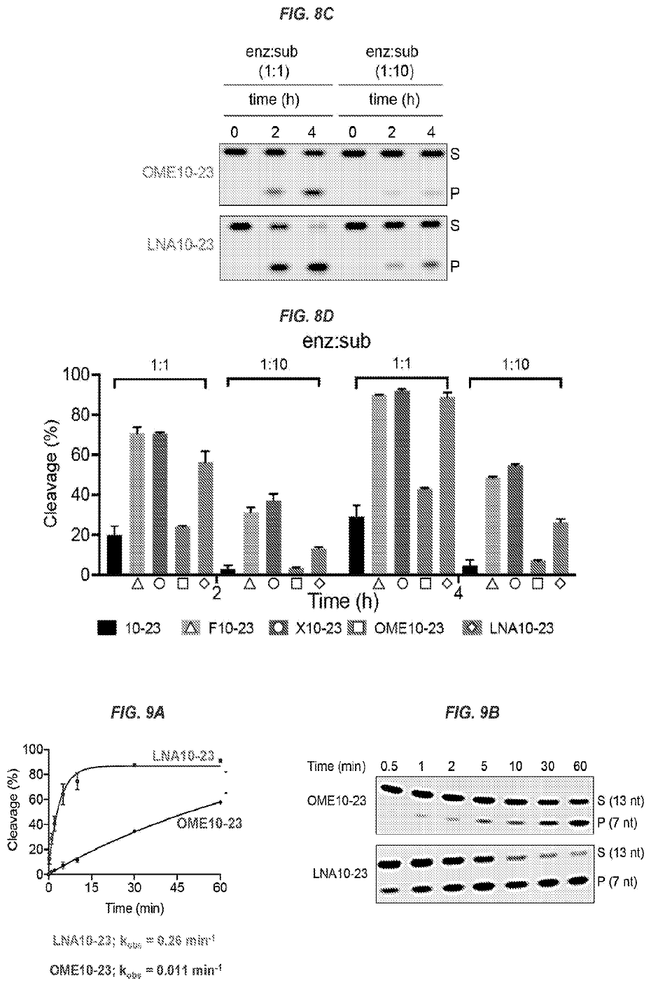

two 10-23 analogs with substrate binding arms that are complementary to the RNA substrate (FIGS. 8A and 8B), were synthesized. OME10-23 is an analog of 10-23 in which 16 DNA residues are replaced with 2′-O-methyl ribonucleotides (bolded-10 in the substrate binding arms and 6 in the catalytic core (underlined)), while LNA10-23 is a 10-23 analog in which three terminal DNA residues in each binding arm are replaced with LNA (replaced residues are bolded and the catalytic core is underlines in the aforementioned sequences).

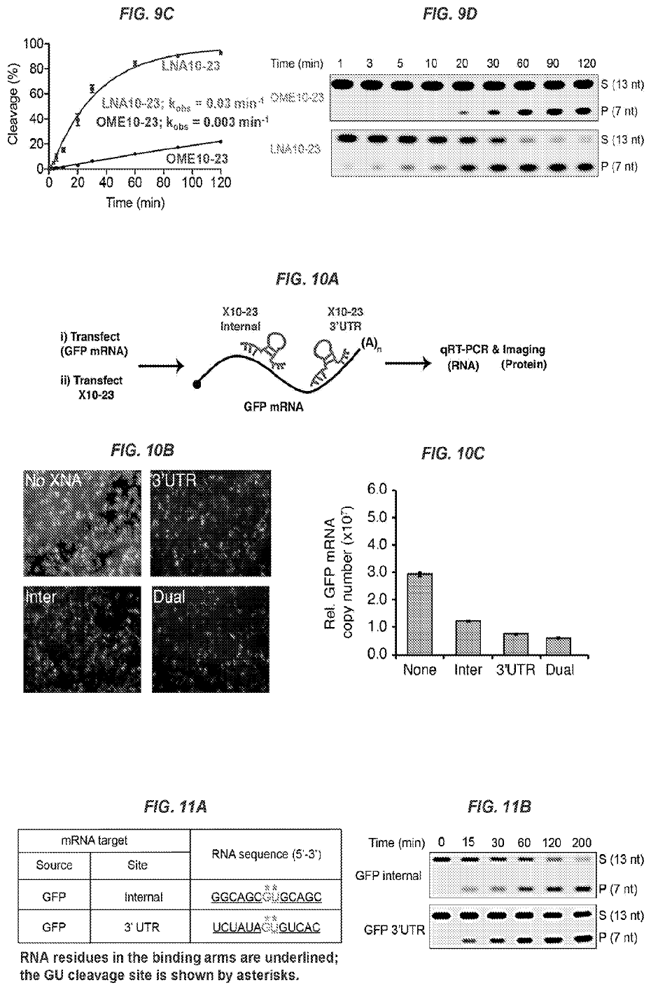

Kinetic measurements indicate that LNA10-23 is significantly faster than OME10-23 under all conditions tested. Under single turnover conditions, LNA10-23 functions with a rate of 0.26 min-1 in the presence of 10 mM MgCl₂ and 0.03 min-1 when the concentration of Mg²⁺ is reduced to 1 mM (FIGS. 9A, 9B, 9C, and 9D). These values compare favorably against OME10-23, which achieves rates of only 0.011 and 0.001 min-1 under identical conditions of high and low Mg²⁺ ions, respectively. The superior activity of LNA10-23 over OME10-23 is maintained under steady-state conditions where substrate and enzyme are present in equimolar concentrations (FIG. 8C). However, the kinetic profile changes dramatically under multiple turnover conditions where LNA10-23 shows clear signs of product inhibition (FIG. 8C). Thus, even though LNA10-23 is a faster enzyme than X10-23 under single turnover conditions, X10-23 is a better candidate for cellular applications (FIG. 8D) due to its robust multiple turnover activity in vitro.

### Intracellular Reduction of GFP:

Next, the activity of X10-23 in cultured mammalian cells was determined using the green fluorescent protein (GFP) as an optical reporter for gene silencing activity. GFP expression was measured in the presence and absence of two X10-23 reagents that were designed to target G-U dinucleotides in the coding (internal) and 3′ untranslated region (3′UTR) of the GFP mRNA transcript (FIG. 10A). Both reagents were independently validated in vitro using synthetic RNA oligonucleotides that matched the GFP segments targeted in the cellular assays (FIGS. 11A and 11B). Cellular assays were performed in multiple formats using HEK293T cells that were transfected with a GFP expression plasmid driven by a CMV promoter. Fluorescent images collected after 24 hours of incubation post-transfection reveal a significant loss of GFP signal for cells that were co-transfected with the GFP plasmid and an X10-23 reagent targeting either the internal site or 3′UTR site, or both sites simultaneously, as compared to cells transfected with the GFP plasmid only (FIG. 10B). qRT-PCR measurements confirm that loss of the GFP signal is due to a drop in mRNA template copy number for GFP (FIG. 10C), demonstrating a reduction of both protein and mRNA levels in the cell.

Without wishing to limit the present invention to any theory or mechanism it was thought that given the strength of the CMV promoter, each X10-23 reagent must be engaging multiple mRNA templates in the cytoplasm in order to maintain strong gene silencing activity under constitutive GFP expression conditions.

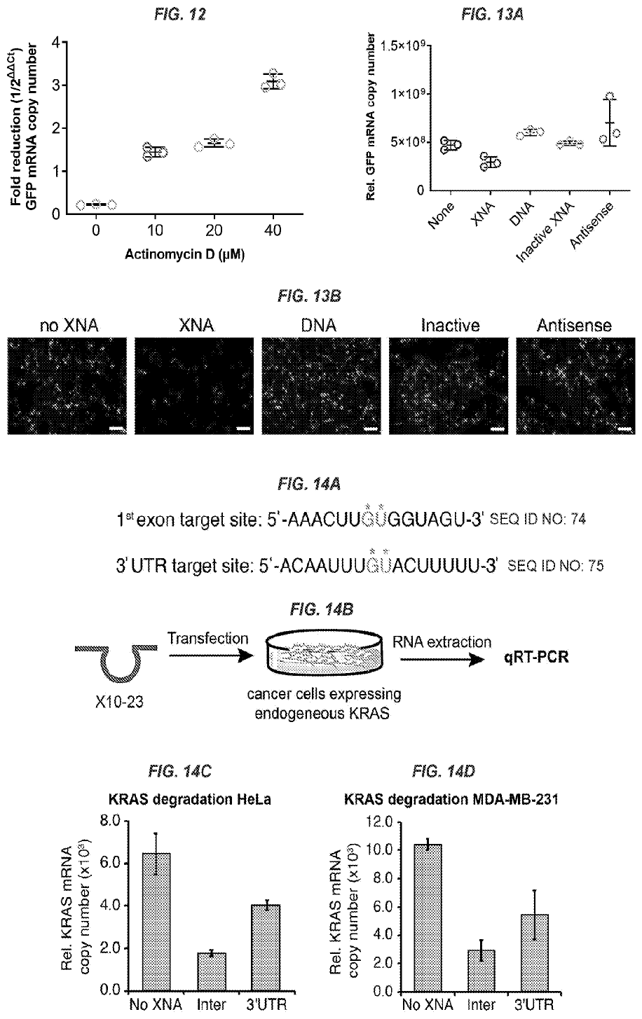

Recognizing that constitutive gene expression from a CMV promoter produces larger quantities of RNA than endogenous gene expression, a dose-dependent treatment of actinomycin D for 4 hours was administered after 20 hours of incubation post-transfection to inhibit RNA transcription. Actinomycin D is a transcriptional inhibitor that prevents continued expression of GFP in the cell, allowing X10-23 to engage only those GFP transcripts that are present when the antibiotic is administered to the cells. qRT-PCR analysis of cellular GFP transcripts shows a 3-fold reduction in template copy number by X10-23 when the cells are treated with 40 μM of actinomycin D, as compared to cells that are co-transfected with the GFP plasmid and X10-23 but not treated with the antibiotic (FIG. 12). These results compare favorably against the parent 10-23 DNA enzyme synthesized with an 3′ inverted dT cap (FIG. 7) as well as the inactive X10-23 variant and a fully complementary FANA antisense strand capped at the 5′ and 3′ ends by unmatched terminal tT residues (FIGS. 13A and 13B). Of the controls, the inactive X10-23 enzyme shows no reduction in GFP protein and mRNA template copy number, while the parent DNAzyme and antisense strand yield slightly elevated values of GFP mRNA and protein (FIGS. 13A and 13B).

### Intracellular Reduction of Endogenous KRAS

Having demonstrated that X10-23 is capable of knocking down the expression of transiently transfected genes in cultured cells, whether similar effects could be achieved for endogenous mRNA transcripts was determined next. KRAS was chosen as a cellular target due to its implication in lung, pancreatic, and colorectal adenocarcinomas. KRAS has been the focus of many drug targeting campaigns and is often viewed as an “undruggable” target due to the inherent difficulty of altering its cellular expression profile. Two X10-23 reagents were designed, synthesized, and tested for targeting the 1st exon and 3′UTR (FIG. 14A) of endogenous KRAS in cervical cancer (HeLa) and breast cancer (MDA-MB-231) cell lines. HeLa and MDA-MB-231cells were either transfected with or without 4 μg of X10-23 and KRAS mRNA levels were quantified by qRT-PCR after 48 hours of incubation post-transfection (FIG. 14B). Relative to the transfection control, both cell lines show a >65% reduction of mRNA copy number for the X10-23 reagent targeting the 1st exon (FIGS. 14C and 14D). The X10-23 reagent targeting 3′UTR was slightly less effective, yielding a ˜35-45% reduction in KRAS mRNA copy number. Differences in the RNA cleavage activity between the two X10-23 reagents is congruent with their in vitro activity observed with synthetic oligonucleotides (FIGS. 15A and 15B), and likely reflects sequence-specific differences in the binding energetics of the two reagents. Overall, these data clearly demonstrate that X10-23 can be used to knockdown the expression of disease-causing proteins in human cells.

Mechanistic Insights into Cellular Cleavage:

In vitro RNAse H activity assay with X10-23, unmatched X10-23, and inactive X10-23: All RNase H activity assays were performed under simulated physiological buffer conditions in 50 mM Tris-HCl (pH 7.5) containing 0.5 mM MgCl₂, 150 mM NaCl, and 0.1 unit/μL of RNase H at 37° C. 1 μM of RNA substrate was mixed with 1 μM of the tested constructs of X10-23, unmatched X10-23, and inactive X10-23 in Tris-HCl (pH 7.5) buffer, respectively, to anneal by heating for 5 min at 90° C. and cooling for 5 min on ice. Reactions were initiated by the addition of MgCl₂, NaCl, and RNase H to the final concentration. Reactions were sampled by quenching 1.5 μL of reactions using 15 μL (10 equivalents, v/v) of formamide containing 25 mM EDTA at time points of 0, 1, 5, and 20 hours. Samples were denatured for 15 min at 95° C. and analyzed by 15% denaturing PAGE. Gels were visualized using a LI-COR Odyssey CLx.

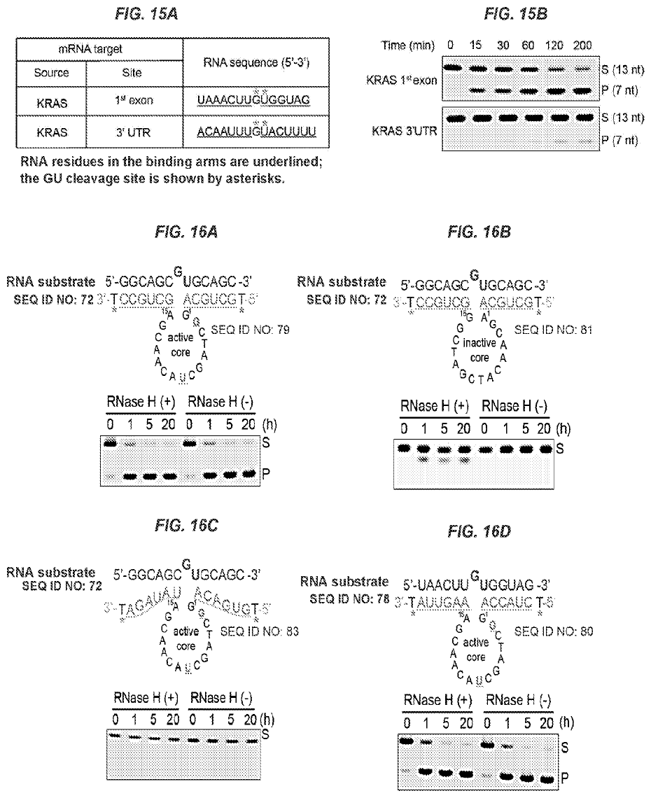

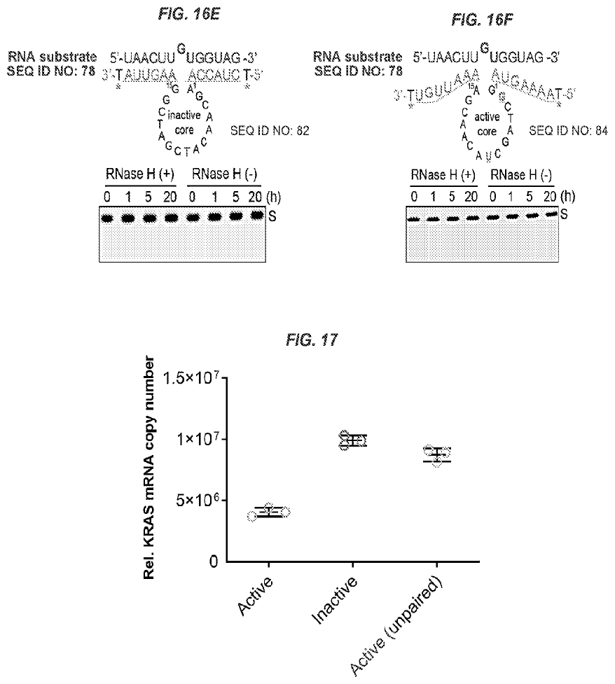

RNase H has been implicated as a contributor to RNA-based degradation by 10-23 variants due to the presence of complementary substrate binding arms that mimic antisense oligonucleotides. Recognizing the substantial difference in multiple turnover activity between 10-23 and X10-23, it was hypothesized that the newly engineered X10-23 reagent was sufficiently fast that it would be less susceptible to the effects of an RNase H induced cleavage pathway. To investigate this possibility, the catalytic activity of X10-23 was evaluated under simulated physiological conditions in buffered solutions that either contain or lack RNase H. X10-23 variants were designed to cleave segments of GFP and KRAS transcripts that were prepared as synthetic oligonucleotides. Inactive versions of X10-23 and active versions that were non-complementary to the mRNA targets were used as negative controls. The X10-23 reagents show strong site-specific RNA cleavage activity in the presence and absence of RNase H (FIGS. 16A and 16D), implicating Mg²⁺-dependent XNAzyme catalyzed RNA cleavage as the predominant mechanism of RNA degradation. Among the negative control sequences, the inactive X10-23 variant targeting GFP yields a banding pattern consistent with limited RNase H activity, while the equivalent KRAS targeting reagent shows no activity in the presence or absence of RNase H (FIGS. 16B and 16E). This observation may be attributed to differences in the hybridization efficiency of the two X10-23 reagents, or possibly, some unknown sequence specificity preference of RNase H. As expected, the second X10-23 control with non-complementary binding arms but an active catalytic core fails to cut the RNA GFP and KRAS RNA substrates (FIGS. 16C and 16F). Similar results were obtained for in vivo GFP and KRAS gene silencing experiments performed in mammalian cells comparing the active X10-23 reagent to the inactive or unpaired control reagents.

TABLE 3

| | Sequence | SEQ ID NO: |
|---|---|---|
| OME10-23 | UCAUGAGGCTAGCUACAACGAGGUUAG | 52 |
| LNA10-23 | TCATGAGGCTAGCTACAACGAGGTTAG | 53 |
| X10-23 that targets GFP (FIG. | ^{t}TgcugcaGgCTAGCuACAACGAgcugcc^{t}T | 46 |
| 16A) | | |
| X10-23 with an inactive core | TGCUGCAGGCTAGCTACAACGAGCUGCC^{t}T | 54 |
| that targets GFP (FIG. 16B) | | |
| X10-23 with no hybridization | ^{t}TgugacaGgCTAGCuACAACGAuauaga^{t}T | 47 |
| to GFP (FIG. 16C) | | |
| X10-23 that targets KRAS | ^{t}TCUACCAGGCTAGCUACAACGAAAGUUA^{t}T | 55 |
| (FIG. 16D) | | |
| X10-23 with an inactive core | ^{t}TCUACCAGGCTAGCTACAACGAAAGUUA^{t}T | 56 |
| that targets KRAS (FIG. 16E) | | |
| X10-23 with no hybridization | ^{t}TAAAAGUAGGCTAGCUACAACGAAAAUUG | 57 |
| to KRAS (FIG. 16F) | U^{t}T | |

Note:
Catalytic domain = underlined; FANA = bolded; TNA = ^{t}; LNA = italicized

The present invention aims to narrow the gap between DNAzymes and protein-based gene silencing tools by expanding the chemical space of nucleic acid analogs used to construct nucleic acid enzymes. Efforts were focused on xeno-nucleic acids, which are artificial genetic polymers with novel sugar-phosphate backbones that harbor unique physicochemical properties relative to natural DNA and RNA.

Without wishing to limit the present invention to any particular theory or mechanism it is thought that appropriate positioning of XNA residues in the nucleic acid backbone structure of a highly evolved DNAzyme will lead to enhanced RNA cleavage activity under physiological conditions, while simultaneously protecting the molecule from the harmful effects of nuclease digestion. This hypothesis is supported by limited structural data showing subtle yet important differences in the helical geometry of XNA duplexes.

Critical to the design of the present invention was the need to identify XNA residues that would (i) increase substrate binding kinetics without sacrificing multiple turnover activity, (ii) improve cofactor binding, and (iii) minimize the exolytic activity of biological enzymes. Using a medicinal chemistry approach that systematically probed the substrate binding arms and catalytic domain of the classic DNAzyme 10-23, a highly efficient and biologically stable variant that comprises three different classes of nucleic acid molecules (DNA, FANA, and TNA) was discovered. Relative to the parent enzyme, X10-23 achieves a ˜50-fold increase in multiple turnover activity under simulated physiological conditions and enhances the biological stability of the backbone structure >100-fold under stringent nuclease conditions. In cultured mammalian cells, X10-23 imbues a >60% reduction in mRNA and protein abundance under conditions of constitutive expression, which is further enhanced upon treatment with a transcriptional inhibitor. Similar activity profiles were observed for X10-23 reagents targeting endogenous KRAS expression in human cancer cell lines, implying that X10-23 has the potential to alter the expression profiles of proteins that are thought to be “undruggable”. Finally, compelling evidence was provided showing that X10-23 does not rely on RNase H as a mechanism for RNA degradation.

In summary, the present invention establishes X10-23 as a new tool in the ever expanding toolbox of gene silencing reagents. The ability for X10-23 to function with high activity and biological stability in vitro and in cultured mammalian cells suggests that even highly evolved nucleic acid enzymes can be optimized for improved activity. Based on these findings, the exploration of new molecular chemotypes provide a powerful approach for creating highly active nucleic acid enzymes with potential value as future therapeutic agents.

### Intracellular GFP and KRAS Reduction Test:

Cell lines and mammalian cell cultures and conditions: HEK293T (HEK) and Hela cells were cultured in DMEM (Corning, Cat #: 10-017-CM) supplemented with 10% FBS, 1% (1 mg/mL) penicillin and streptomycin and grown at 37° C., 5% CO₂. MDA-MB-231 cells were cultured in the same medium as HEK and HeLa cells but supplemented with additional components of 1 mM sodium pyruvate.

Transfection: For titrating amount of single or multivalent X10-23 experiments: After 48 h seeding of 2.5×10⁵ cells/well, HEK293T in 6-well plates were transfected with 1 μg of pCDNA3.3-EGFP only (Negative control) or with 1 μg of pCDNA3.3-EGFP and 4, 6, or 8 μg of either internal or 3′UTR GFP X10-23 in single experiments or 2/2 μg, 3/3 μg or 4/4 μg of both internal/3′UTR GFP X10-23 in multivalent (dual X10-23) experiments by using JetPrime Transfection reagent (Polyplus Transfection, France) according to manual instruction except adding 5× higher than the manual recommended volume of JetPrime Reagent. For negative controls, the volume of JetPrime Reagent used for each well was the same as those with X10-23 to ensure the same transfection condition in the control and experimental samples

For comparison of active, inactive core and active unpaired X10-23: After 48 h seeding at 2.5×10⁵ cells/well (6-well plate), Hela cells were transfected with 5.9 μg×10-23 variants targeting the 3′UTR region of KRAS transcript (active vs. inactive core) or GFP transcript (active core but unpaired binding arms) using JetPrime Transfection reagent. At 96 h post transfection, cells were harvested and subjected to total RNA extraction and subsequently underwent DNAse treatment as described in the RNA isolation section. DNA-free RNA was subjected to RT-qPCR as described in the reverse transcription and SYBR Green qPCR analysis section.

For multivalent benchmark experiments: After 48 h seeding at 2.5×10⁵ cells/well, HEK293T in 6-well plates were transfected with 1 μg of pCDNA3.3-EGFP only (Negative control) or with 1 μg of pCDNA3.3-EGFP and 4/4 μg of both internal/3′UTR GFP X10-23 (dual X10-23), DNA10-23 (dual DNA10-23), antisense (dual antisense oligos), inactive X10-23 (dual Inactive X10-23) or inactive DNA10-23 (dual inactive DNA10-23). Parameters used in subsequent imaging and RNA isolation at 48 h post-transfection are the same as described in RNA isolation section.

For single or multivalent KRAS X10-23 experiments in HeLa or MDA-MB-231 cells: After 48 h, 76 h seeding of 2.5×10⁵ cells/well of HeLa or MDA-MB-231 cells in 6-well plates, respectively, the cells were transfected with transfection carrier only (Negative control) or with 4/4 μg of both internal/3′UTR KRAS X10-23 in multivalent (dual X10-23) or 8 μg of either Internal or 3′UTR KRAS X10-23 experiments using JetPrime Transfection reagent (Polyplus Transfection, France). Parameters used in RNA isolation at 48 h post-transfection are the same as described in the RNA isolation section.

Cell imaging: At 24 h or 48 h post-transfection, cells were subjected to live imaging using 200M Axiovert Zeiss fluorescent microscope with 10× objective and GFP filter. Following imaging, the cells were subjected to RNA extraction.

Reverse transcription (RT): Two micrograms (2 μg) of DNA-free RNA were subjected to cDNA synthesis using SuperScript III First-strand Synthesis System (Invitrogen-Life Technologies, CA) with random hexamer primers in a 20 μL reaction according to the manufacturer instructions. cDNA was subsequently purified using DNA Clean & Concentration columns from Zymo Research (Cat #D4003) according to the manufacturer instructions and eluted 2× with 100 μL water/each.

SYBR Green semi-quantitative PCR (qPCR) analysis. To quantify copy number of GFP transcript in the presence or absence of GFP-X10-23, cDNA was subjected to qPCR analysis using iQ(tm) SYBR(R) Green Supermix (BioRad, Cat #1708880) on BioRad CFX real time PCR system. In each qPCR run, known concentration of DNA standards at 5 serial dilutions was used to establish standard curve and calculation of starting quantity (SQ) of target transcripts. Specific primers for interrogating EGFP, KRAS, GAPDH (loading control) transcripts as well as for qPCR Standards are listed in Table 4. For each experimental sample, three replicates were performed, and each serial diluted Standard was assayed in duplicates. Relative mRNA copy number of target transcripts was calculated by multiplying individual starting quantity (SQ) to a corresponding scaling factor derived from loading control GAPDH SQ. By dividing the median of GAPDH SQ in a qPCR run to individual GAPDH SQ, a scaling factor for that particular sample was generated. Fold reduction was calculated using 1/2^{ΔΔCt}.

TABLE 4
Table of Primers used for qRT-PCR

| SEQ | | | |
|---|---|---|---|
| ID | Primer | | |
| NO: | ID | Sequence | Target |
| 58 | oKN60F | 5′-ACC ATC TTC CAG GAG | GAPDH |
| | | CGA GAT CCC TC-′3 | |
| 59 | oKN61R | 5′-TGC AGG AGG CAT TGC | GAPDH |
| | | TGA TGA TCT TGA-′3 | |
| 60 | oKN111FC | 5′-ACCATGAGTG ATAACACTGC | Standard |
| | | GGC-′3 | |
| 61 | OKN112RC | 5′-CGG TTC CCA ACG ATC | Standard |
| | | AAG GC-′3 | |
| 62 | oKN424F | 5′-CAG CCA CAA CGT CTA | EGFP |
| | | TAT CAT GGC CG-′3 | |
| 63 | oKN421R | 5′-GCT CAG GTA GTG GTT | EGFP |
| | | GTC GGG CAG-′3 | |
| 64 | oKN469F | 5′-GGC CTG CTG AAA ATG | KRAS |
| | | ACT GAA TAT AAA C-′3 | |
| 65 | oKN470R | 5′-ACA AGA TTT ACC TCT | KRAS |
| | | ATT GTT GGA TCA TAT TCG-′3 | |

RNA Polymerase inhibitor (Actinomycin D, ActD) treatment. In titration of ActD concentration experiment, cultures of HEK293T cells in 6-well plates transfected with either 1 μg of pCDNA3.3-EGFP only (Negative control) or with 1 μg of pCDNA3.3-EGFP and 4 μg of Internal GFP X10-23 were treated with ActD at final concentration of 0, 10, 20, 40 μM at 20 h post transfection and harvested at 4 h later (at 24 h post transfection). Treated cells were harvested and subjected to total RNA isolation and subsequent analyses. In benchmark experiment, 40 μM ActD was added to HEK293T cells transfected with dual 4 μg Internal and 4 μg 3′UTR of X10-23, 10-23, Inactive X10-23 or antisense at 44 h post transfection and incubated at 37° C. for additional 4 h prior harvesting time at 48 h post transfection. Treated cells were imaged and subjected to total RNA isolation and subsequent analyses.

RNA isolation. To each well of the 6-well plate of cells (HEK293T, HeLa or MDA-MB-231), total RNA isolation was performed using 1 mL/well Trizol Reagent (Invitrogen) according to the manufacturer instructions. Total RNA was treated with Turbo DNAse (20 U/reaction) at 37° C. for 30 min on shaker, and followed by purification using equal volume of Phenol-Chloroform, pH 4.5 (Thermal Fisher, Ambion Cat #: AM9720). Aqueous layer was transferred to a new tube and precipitated with one tenth volume of 5 M NaCl and one volume of isopropanol at −20° C. overnight. Precipitated RNA was pelleted at 4° C. and 15000 rpm using bench-top centrifuge and followed by two washes with cold (−20° C.) 70% ethanol

General Information: 2′F-araNTPs (faATP, faCTP, faGTP, faUTP) were obtained from Metkinen Chemistry (Kuusisto, Finland). DNA, FANA, and 5′-hexynyl phosphoramidites, as well as Universal Support II CPG columns were purchased from Glen Research (Sterling, Virginia). TNA phosphoramidites were synthesized in-house following procedures reported previously in the art. Oligonucleotides containing FANA and TNA were synthesized on an ABI3400 DNA synthesizer using chemical synthesis reagents purchased from Glen Research (Sterling, Virginia). DNA and RNA oligonucleotides were purchased from Integrated DNA Technologies (Coralville, IA). All oligonucleotides were purified by denaturing polyacrylamide gel electrophoresis and quantified by UV absorbance. YM-3 microcentrifugal concentrators were purchased from EMD Millipore (Billerica, MA). Dulbecco's Modified Eagle Medium (DMEM) was purchased from ThermoFisher Scientific (Waltham, MA). Human serum and snake venom phosphodiesterase were purchased from Sigma Aldrich (St. Louis, MO). Human liver microsome was purchased from Sekisui XenoTech, LLC.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A composition for gene silencing, the composition comprising:
a) a 15-nucleotide catalytic domain according to SEQ ID NO: 1, wherein one or more nucleic acids of the catalytic domain is replaced by xeno-nucleic acids (XNA);
b) a first substrate recognition domain 5′ to the catalytic domain;
c) a second substrate recognition domain 3′ to the catalytic domain;
d) a 5′ terminal threose nucleic acid (TNA) residue; and
e) a 3′ terminal TNA residue;
wherein one or more nucleic acids of the first substrate recognition domain or the second recognition domain is replaced by XNA; and
wherein the composition has enhanced stability and enhanced catalytic activity compared to a control molecule comprising wild type SEQ ID NO: 1 as its catalytic domain.

2. The composition of claim 1, wherein the XNA is 2′-fluoroarabino nucleic acid (FANA).

3. The composition of claim 1, wherein the XNA is TNA.

4. The composition of claim 1, wherein the composition is for knocking down a target RNA.

5. The composition of claim 4, wherein the target RNA is KRAS.

6. The composition of claim 1, wherein the first substrate recognition domain and second substrate recognition domain are at least 5 nucleotides long.

7. A method of treating a disease or condition or a symptom thereof, the method comprising administering an effective amount of a 10-23 analogue composition to a subject in need thereof, wherein the composition comprises:
a) a 15-nucleotide catalytic domain according to SEQ ID NO: 1, wherein one or more nucleic acids of the catalytic domain is replaced by xeno-nucleic acids (XNA);
b) a first substrate recognition domain 5′ to the catalytic domain;
c) a second substrate recognition domain 3′ to the catalytic domain; and
d) a 5′ terminal threose nucleic acid (TNA) residue; and
e) a 3′ terminal TNA residue;
wherein one or more nucleic acids of the first substrate recognition domain or the second recognition domain is replaced by XNA.

8. The method of claim 7, wherein the XNA is 2′-fluoroarabino nucleic acid (FANA).

9. The method of claim 7, wherein the XNA is TNA.

10. The method of claim 7, wherein the composition is for knocking down a target RNA.

11. The method of claim 10, wherein the target RNA is KRAS.

12. The method of claim 7, wherein the first substrate recognition domain and second substrate recognition domain are at least 5 nucleotides long.

13. The method of claim 7, wherein the disease or condition is caused by a common or rare genetic disease, viral or bacterial pathogen, cancer, inflammation, cardiovascular disease, immune deficiency, or a neurological disorder.

14. A method of validating gene mutations associated with a disease or condition, the method comprising:
a) administering a 10-23 analogue composition to a cell line or animal model, wherein the composition comprises:
i) a 15-nucleotide catalytic domain according to SEQ ID NO: 1, wherein one or more nucleic acids of the catalytic domain is replaced by xeno-nucleic acids (XNA);
ii) a first substrate recognition domain 5′ to the catalytic domain;
iii) a second substrate recognition domain 3′ to the catalytic domain;
iv) a 5′ terminal threose nucleic acid (TNA) residue; and
v) a 3′ terminal TNA residue;
wherein one or more nucleic acids of the first substrate recognition domain or the second recognition domain is replaced by XNA; and
b) analyzing the cell line for characteristics associated with the disease or condition.

15. The method of claim 14, wherein the XNA is 2′-fluoroarabino nucleic acid (FANA).

16. The method of claim 14, wherein the XNA is TNA.

17. The method of claim 14, wherein the composition mutates a target RNA.

18. The method of claim 14, wherein the target RNA is KRAS.

19. The method of claim 14, wherein the first substrate recognition domain and second substrate recognition domain are at least 5 nucleotides long.

20. The method of claim 14, wherein the disease or condition is caused by a common or rare genetic disease, viral or bacterial pathogen, cancer, inflammation, cardiovascular disease, immune deficiency, or a neurological disorder.


SEQUENCE LISTING (84 sequences)

SEQ ID NO: 1 - DNA, 15 bp, Unknown
  XNAzymes Catalytic Domain
ggctagctacaacga

SEQ ID NO: 2 - DNA, 15 bp, Unknown
  XNAzymes Catalytic Domain Sequence
  misc_feature (1)..(1): FANA residue
ggctagctacaacga

SEQ ID NO: 3 - DNA, 15 bp, Unknown
  XNAzymes Catalytic Domain Sequence
  misc_feature (2)..(2): FANA residue
ggctagctacaacga

SEQ ID NO: 4 - DNA, 15 bp, Unknown
  XNAzymes Catalytic Domain Sequence
  misc_feature (3)..(3): FANA residue
ggctagctacaacga

SEQ ID NO: 5 - DNA, 15 bp, Unknown
  XNAzymes Catalytic Domain Sequence
  misc_feature (4)..(4): FANA residue
ggctagctacaacga

SEQ ID NO: 6 - DNA, 15 bp, Unknown
  XNAzymes Catalytic Domain Sequence
  misc_feature (5)..(5): FANA residue
ggctagctacaacga

SEQ ID NO: 7 - DNA, 15 bp, Unknown
  XNAzymes Catalytic Domain Sequence
  misc_feature (6)..(6): FANA residue
ggctagctacaacga

SEQ ID NO: 8 - DNA, 15 bp, Unknown
  XNAzymes Catalytic Domain Sequence
  misc_feature (7)..(7): FANA residue
ggctagctacaacga

SEQ ID NO: 9 - DNA, 15 bp, Unknown
  XNAzymes Catalytic Domain Sequence
  misc_feature (8)..(8): FANA residue
ggctagctacaacga

SEQ ID NO: 10 - DNA, 15 bp, Unknown
  XNAzymes Catalytic Domain Sequence
  misc_feature (9)..(9): FANA residue
ggctagctacaacga

SEQ ID NO: 11 - DNA, 15 bp, Unknown
  XNAzymes Catalytic Domain Sequence
  misc_feature (10)..(10): FANA residue
ggctagctacaacga

SEQ ID NO: 12 - DNA, 15 bp, Unknown
  XNAzymes Catalytic Domain Sequence
  misc_feature (11)..(11): FANA residue
ggctagctacaacga

SEQ ID NO: 13 - DNA, 15 bp, Unknown
  XNAzymes Catalytic Domain Sequence
  misc_feature (12)..(12): FANA residue
ggctagctacaacga

SEQ ID NO: 14 - DNA, 15 bp, Unknown
  XNAzymes Catalytic Domain Sequence
  misc_feature (13)..(13): FANA residue
ggctagctacaacga

SEQ ID NO: 15 - DNA, 15 bp, Unknown
  XNAzymes Catalytic Domain Sequence
  misc_feature (14)..(14): FANA residue
ggctagctacaacga

SEQ ID NO: 16 - DNA, 15 bp, Unknown
  XNAzymes Catalytic Domain Sequence
  misc_feature (15)..(15): FANA residue
ggctagctacaacga

SEQ ID NO: 17 - DNA, 15 bp, Unknown
  XNAzymes Catalytic Domain Sequence
  misc_feature (2)..(2): FANA residue
  misc_feature (8)..(8): FANA residue
ggctagctacaacga

SEQ ID NO: 18 - DNA, 15 bp, Unknown
  XNAzymes Catalytic Domain Sequence
  misc_feature (2)..(2): FANA residue
  misc_feature (4)..(4): FANA residue
  misc_feature (8)..(8): FANA residue
  misc_feature (9)..(9): FANA residue
ggctagctacaacga

SEQ ID NO: 19 - DNA, 27 bp, Unknown
  F10-23 (6 nt binding arms)
  misc_feature (1)..(6): FANA residues
  misc_feature (22)..(27): FANA residues
ucaugaggctagctacaacgagguuag

SEQ ID NO: 20 - DNA, 27 bp, Unknown
  10-23 (6 nt binding arms)
tcatgaggctagctacaacgaggttag

SEQ ID NO: 21 - DNA, 29 bp, Unknown
  10-23 (7 nt binding arms)
gtcatgaggctagctacaacgaggttagg

SEQ ID NO: 22 - RNA, 13 nt, Unknown
  RNA substrate (6 nt binding arms)
cuaaccgucauga

SEQ ID NO: 23 - RNA, 15 nt, Unknown
  RNA substrate (7 nt binding arms)
ccuaaccgucaugac

SEQ ID NO: 24 - DNA, 29 bp, Unknown
  F10-23 V2 (7 nt binding arms)
  misc_feature (1)..(7): FANA residues
  misc_feature (23)..(29): FANA residues
gcacccaggctagctacaacgacucucuc

SEQ ID NO: 25 - DNA, 29 bp, Unknown
  10-23 V2 (7 nt binding arms)
gcacccaggctagctacaacgactctctc

SEQ ID NO: 26 - RNA, 15 nt, Unknown
  RNA substrate V2  (7 nt binding arms)
gagagaggugggugc

SEQ ID NO: 27 - RNA, 103 nt, Unknown
  103 nt RNA Containing target site
ccuuuucuuaauuugauuccugccaacacccuaaccgucaugacuucaccuuuuaccauggguggcuuauuaaaaacauuuuccuggacgaagacaguucccu

SEQ ID NO: 28 - DNA, 27 bp, Unknown
  10-23 G1
  misc_feature (7)..(7): FANA residue
tcatgaggctagctacaacgaggttag

SEQ ID NO: 29 - DNA, 27 bp, Unknown
  10-23 G2
  misc_feature (8)..(8): FANA residue
tcatgaggctagctacaacgaggttag

SEQ ID NO: 30 - DNA, 27 bp, Unknown
  10-23 C3
  misc_feature (9)..(9): FANA residue
tcatgaggctagctacaacgaggttag

SEQ ID NO: 31 - DNA, 27 bp, Unknown
  10-23 U4
  misc_feature (10)..(10): FANA residue
tcatgaggcuagctacaacgaggttag

SEQ ID NO: 32 - DNA, 27 bp, Unknown
  10-23 A5
  misc_feature (11)..(11): FANA residue
tcatgaggctagctacaacgaggttag

SEQ ID NO: 33 - DNA, 27 bp, Unknown
  10-23 G6
  misc_feature (12)..(12): FANA residue
tcatgaggctagctacaacgaggttag

SEQ ID NO: 34 - DNA, 27 bp, Unknown
  10-23 C7
  misc_feature (13)..(13): FANA residue
tcatgaggctagctacaacgaggttag

SEQ ID NO: 35 - DNA, 27 bp, Unknown
  10-23 U8
  misc_feature (14)..(14): FANA residue
tcatgaggctagcuacaacgaggttag

SEQ ID NO: 36 - DNA, 27 bp, Unknown
  10-23 A9
  misc_feature (15)..(15): FANA residue
tcatgaggctagctacaacgaggttag

SEQ ID NO: 37 - DNA, 27 bp, Unknown
  10-23 C10
  misc_feature (16)..(16): FANA residue
tcatgaggctagctacaacgaggttag

SEQ ID NO: 38 - DNA, 27 bp, Unknown
  10-23 A11
  misc_feature (17)..(17): FANA residue
tcatgaggctagctacaacgaggttag

SEQ ID NO: 39 - DNA, 27 bp, Unknown
  10-23 A12
  misc_feature (18)..(18): FANA residue
tcatgaggctagctacaacgaggttag

SEQ ID NO: 40 - DNA, 27 bp, Unknown
  10-23 C13
  misc_feature (19)..(19): FANA residue
tcatgaggctagctacaacgaggttag

SEQ ID NO: 41 - DNA, 27 bp, Unknown
  10-23 G14
  misc_feature (20)..(20): FANA residue
tcatgaggctagctacaacgaggttag

SEQ ID NO: 42 - DNA, 27 bp, Unknown
  10-23 A15
  misc_feature (21)..(21): FANA residue
tcatgaggctagctacaacgaggttag

SEQ ID NO: 43 - DNA, 27 bp, Unknown
  10-23 G2/U8
  misc_feature (8)..(8): FANA residue
  misc_feature (14)..(14): FANA residue
tcatgaggctagcuacaacgaggttag

SEQ ID NO: 44 - DNA, 27 bp, Unknown
  10-23 G2/U4/U8/A9
  misc_feature (8)..(8): FANA residue
  misc_feature (10)..(10): FANA residue
  misc_feature (14)..(14): FANA residue
  misc_feature (15)..(15): FANA residue
tcatgaggcuagcuacaacgaggttag

SEQ ID NO: 45 - DNA, 29 bp, Unknown
  X10-23
  misc_feature (1)..(1): TNA residue
  misc_feature (2)..(7): FANA residues
  misc_feature (9)..(9): FANA residues
  misc_feature (15)..(15): FANA residues
  misc_feature (23)..(28): FANA residues
  misc_feature (29)..(29): TNA residue
tucaugaggctagcuacaacgagguuagt

SEQ ID NO: 46 - DNA, 29 bp, Unknown
  X10-23 targeting GFP internal
  misc_feature (1)..(1): TNA residue
  misc_feature (2)..(7): FANA residue
  misc_feature (9)..(9): FANA residue
  misc_feature (15)..(15): FANA residue
  misc_feature (23)..(28): FANA residue
  misc_feature (29)..(29): TNA residue
tgcugcaggctagcuacaacgagcugcct

SEQ ID NO: 47 - DNA, 29 bp, Unknown
  X10-23 targeting GFP 3?UTR
  misc_feature (1)..(1): TNA residue
  misc_feature (2)..(7): FANA residue
  misc_feature (9)..(9): FANA residue
  misc_feature (15)..(15): FANA residue
  misc_feature (23)..(28): FANA residue
  misc_feature (29)..(29): TNA residue
tgugacaggctagcuacaacgauauagat

SEQ ID NO: 48 - DNA, 28 bp, Unknown
  10-23 targeting GFP internal
  misc_feature (29)..(29): 3? inverted dT
gctgcaggctagctacaacgagctgcct

SEQ ID NO: 49 - DNA, 28 bp, Unknown
  10-23 targeting GFP 3? UTR
  misc_feature (28)..(28): 3? inverted dT
gtgacaggctagctacaacgatatagat

SEQ ID NO: 50 - DNA, 16 bp, Unknown
  X-antisense targeting
  misc_feature (1)..(1): TNA residue
  misc_feature (16)..(16): TNA residue
ttgcugcacgcugcct

SEQ ID NO: 51 - DNA, 15 bp, Unknown
  X-antisense targeting GFP 3? UTR
  misc_feature (1)..(1): TNA residue
  misc_feature (15)..(15): TNA residue
tgugacacuauagat

SEQ ID NO: 52 - DNA, 27 bp, Unknown
  OME10-23
  misc_feature (1)..(5): FANA residue
  misc_feature (8)..(8): FANA residue
  misc_feature (13)..(14): FANA residue
  misc_feature (17)..(17): FANA residue
  misc_feature (20)..(21): FANA residue
  misc_feature (23)..(27): FANA residue
ucaugaggctagcuacaacgagguuag

SEQ ID NO: 53 - DNA, 27 bp, Unknown
  LNA10-23
  misc_feature (1)..(3): LNA residue
  misc_feature (25)..(27): LNA residue
tcatgaggctagctacaacgaggttag

SEQ ID NO: 54 - DNA, 29 bp, Unknown
  X10-23 with an inactive core that targets
  misc_feature (1)..(1): TNA residue
  misc_feature (2)..(7): FANA residue
  misc_feature (23)..(28): FANA residue
  misc_feature (29)..(29): TNA residue
tgcugcaggctagctacaacgagcugcct

SEQ ID NO: 55 - DNA, 29 bp, Unknown
  X10-23 that targets KRAS
  misc_feature (1)..(1): TNA residue
  misc_feature (2)..(7): FANA residue
  misc_feature (9)..(9): FANA residue
  misc_feature (15)..(15): FANA residue
  misc_feature (23)..(28): FANA residue
  misc_feature (29)..(29): TNA residue
tcuaccaggctagcuacaacgaaaguuat

SEQ ID NO: 56 - DNA, 29 bp, Unknown
  X10-23 with an inactive core that targets KRAS
  misc_feature (1)..(1): TNA residue
  misc_feature (2)..(7): FANA residue
  misc_feature (23)..(28): FANA residue
  misc_feature (29)..(29): TNA residue
tcuaccaggctagctacaacgaaaguuat

SEQ ID NO: 57 - DNA, 31 bp, Unknown
  X10-23 with no hybridization to KRAS
  misc_feature (1)..(1): TNA residue
  misc_feature (2)..(8): FANA residue
  misc_feature (10)..(10): FANA residue
  misc_feature (16)..(16): FANA residue
  misc_feature (24)..(30): FANA residue
  misc_feature (31)..(31): TNA residue
taaaaguaggctagcuacaacgaaaauugut

SEQ ID NO: 58 - DNA, 26 bp, Unknown
  oKN60F primer
accatcttccaggagcgagatccctc

SEQ ID NO: 59 - DNA, 27 bp, Unknown
  oKN61R primer
tgcaggaggcattgctgatgatcttga

SEQ ID NO: 60 - DNA, 23 bp, Unknown
  oKN111FC primer
accatgagtgataacactgcggc

SEQ ID NO: 61 - DNA, 20 bp, Unknown
  oKN112RC primer
cggttcccaacgatcaaggc

SEQ ID NO: 62 - DNA, 26 bp, Unknown
  oKN424F primer
cagccacaacgtctatatcatggccg

SEQ ID NO: 63 - DNA, 24 bp, Unknown
  oKN421R primer
gctcaggtagtggttgtcgggcag

SEQ ID NO: 64 - DNA, 28 bp, Unknown
  oKN469F primer
ggcctgctgaaaatgactgaatataaac

SEQ ID NO: 65 - DNA, 33 bp, Unknown
  oKN470R primer
acaagatttacctctattgttggatcatattcg

SEQ ID NO: 66 - RNA, 13 nt, Unknown
  RNA substrate
cuaaccgucauga

SEQ ID NO: 67 - DNA, 27 bp, Unknown
  10-23
gattggagcaacatcgatcggagtact

SEQ ID NO: 68 - DNA, 27 bp, Unknown
  F10-23 enzyme
gauuggagcaacatcgatcggaguacu

SEQ ID NO: 69 - DNA, 31 bp, Unknown
  X10-23
  misc_feature (1)..(2): TNA residue
  misc_feature (3)..(8): FANA residue
  misc_feature (16)..(16): FANA residue
  misc_feature (22)..(29): FANA residue
  misc_feature (30)..(31): TNA residue
ttgauuggagcaacaucgatcggaguacutt

SEQ ID NO: 70 - RNA, 15 nt, Unknown
  RNA substrate V2
gagagaggugggugc

SEQ ID NO: 71 - DNA, 27 bp, Unknown
  F10-23 V2
  misc_feature (1)..(6): FANA residue
  misc_feature (22)..(27): FANA residue
cucucuagcaacatcgatcggaccacg

SEQ ID NO: 72 - RNA, 13 nt, Unknown
  GFP internal sequence
ggcagcgugcagc

SEQ ID NO: 73 - RNA, 13 nt, Unknown
  GFP 3′UTR sequence
ucuauagugucac

SEQ ID NO: 74 - RNA, 14 nt, Unknown
  first exon of KRAS
aaacuugugguagu

SEQ ID NO: 75 - RNA, 16 nt, Unknown
  3′UTR region of KRAS
acaauuuguacuuuuu

SEQ ID NO: 76 - RNA, 14 nt, Unknown
  first exon KRAS
uaaacuugugguag

SEQ ID NO: 77 - RNA, 15 nt, Unknown
  3′UTR KRAS
acaauuuguacuuuu

SEQ ID NO: 78 - RNA, 13 nt, Unknown
  RNA substrate
uaacuugugguag

SEQ ID NO: 79 - DNA, 29 bp, Unknown
  X10- 23 with an active catalytic core
  misc_feature (1)..(1): TNA residue
  misc_feature (2)..(7): FANA residue
  misc_feature (15)..(15): FANA residue
  misc_feature (21)..(21): FANA residue
  misc_feature (23)..(28): FANA residue
  misc_feature (29)..(29): TNA residue
tccgucgagcaacaucgatcggacgucgt

SEQ ID NO: 80 - DNA, 29 bp, Unknown
  X10- 23 with an active catalytic core
  misc_feature (1)..(1): TNA residue
  misc_feature (2)..(7): FANA residue
  misc_feature (15)..(15): FANA residue
  misc_feature (21)..(21): FANA residue
  misc_feature (23)..(28): FANA residue
  misc_feature (29)..(29): TNA residue
tauugaaagcaacaucgatcggaccauct

SEQ ID NO: 81 - DNA, 29 bp, Unknown
  X10-23 with an inactive catalytic core
  misc_feature (1)..(1): TNA residue
  misc_feature (2)..(7): FANA residue
  misc_feature (23)..(28): FANA residue
  misc_feature (29)..(29): TNA residue
tccgucgggctagctacaacgaacgucgt

SEQ ID NO: 82 - DNA, 29 bp, Unknown
  X10-23 with an inactive catalytic core
  misc_feature (1)..(1): TNA residue
  misc_feature (2)..(7): FANA residue
  misc_feature (23)..(28): FANA residue
  misc_feature (29)..(29): TNA residue
tauugaaggctagctacaacgaaccauct

SEQ ID NO: 83 - DNA, 29 bp, Unknown
  X10-23 with an active catalytic core that does not hybridize to the RNA target
  misc_feature (1)..(1): TNA residue
  misc_feature (2)..(7): FANA residue
  misc_feature (15)..(15): FANA residue
  misc_feature (21)..(21): FANA residue
  misc_feature (23)..(28): FANA residue
  misc_feature (29)..(29): FANA residue
tagauauagcaacaucgatcggacagugt

SEQ ID NO: 84 - DNA, 30 bp, Unknown
  X10-23 with an active catalytic core that does not hybridize to the RNA target
  misc_feature (1)..(1): TNA residue
  misc_feature (2)..(7): FANA residue
  misc_feature (15)..(15): FANA residue
  misc_feature (21)..(21): FANA residue
  misc_feature (23)..(29): FANA residue
  misc_feature (30)..(30): FANA residue
tuguuaaagcaacaucgatcggaugaaaat